SEQUENCE LISTING (374 sequences)

SEQ ID NO: 1 - DNA, 21 bp, Mus musculus
aagccgtcctgtgtgccgctg

SEQ ID NO: 2 - DNA, 21 bp, Mus musculus
aacgatgaagccctggagtgc

SEQ ID NO: 3 - DNA, 21 bp, Mus musculus
aagttaaaagtgcctgaactg

SEQ ID NO: 4 - DNA, 21 bp, Mus musculus
aagcaggccagactctctttc

SEQ ID NO: 5 - DNA, 21 bp, Mus musculus
aagctcagcacacagaaagac

SEQ ID NO: 6 - DNA, 21 bp, Mus musculus
aatgcggcggtggtgacagta

SEQ ID NO: 7 - DNA, 10 bp, Artificial Sequence
  Description of Artificial Sequence Synthetic oligonucleotide
tcaacgttga

SEQ ID NO: 8 - DNA, 15 bp, Artificial Sequence
  Description of Artificial Sequence Synthetic oligonucleotide
gctagacgttagcgt

SEQ ID NO: 9 - DNA, 21 bp, Escherichia coli
aacagttgcgcagcctgaatg

SEQ ID NO: 10 - DNA, 21 bp, Escherichia coli
aacttaatcgccttgcagcac

SEQ ID NO: 11 - DNA, 21 bp, Artificial Sequence
  Description of Artificial Sequence Synthetic oligonucleotide
aagctatgaaacgatatgggc

SEQ ID NO: 12 - DNA, 21 bp, Artificial Sequence
  Description of Artificial Sequence Synthetic oligonucleotide
aaccgctggagagcaactgca

SEQ ID NO: 13 - DNA, 47 bp, Artificial Sequence
  Description of Artificial Sequence Synthetic primer
gaacatcgatgacaagcttaggtatcgatacaagctgcctcgccttg

SEQ ID NO: 14 - DNA, 47 bp, Artificial Sequence
  Description of Artificial Sequence Synthetic primer
gaacatcgatgacaagcttaggtatcgatatagattgaagattccgc

SEQ ID NO: 15 - DNA, 47 bp, Artificial Sequence
  Description of Artificial Sequence Synthetic primer
gaacatcgatgacaagcttaggtatcgataggtcactgacagaggcg

SEQ ID NO: 16 - DNA, 30 bp, Artificial Sequence
  Description of Artificial Sequence Synthetic primer
gaacatcgatgacaagcttaggtatcgata

SEQ ID NO: 17 - DNA, 30 bp, Artificial Sequence
  Description of Artificial Sequence Synthetic primer
gatgtctaccagcgaagctactgccgtccg

SEQ ID NO: 18 - DNA, 30 bp, Artificial Sequence
  Description of Artificial Sequence Synthetic primer
gtcagctgctgggacaccgcggtcttgcct

SEQ ID NO: 19 - DNA, 30 bp, Artificial Sequence
  Description of Artificial Sequence Synthetic primer
ggcgctgctagctgtcgctctgtggttctg

SEQ ID NO: 20 - DNA, 20 bp, Artificial Sequence
  Description of Artificial Sequence Synthetic primer
cctggtcaccagggctgctt

SEQ ID NO: 21 - DNA, 20 bp, Artificial Sequence
  Description of Artificial Sequence Synthetic primer
ccagccttctccatggtggt

SEQ ID NO: 22 - DNA, 18 bp, Artificial Sequence
  Description of Artificial Sequence Synthetic primer
gcgggctgcctcgcagtc

SEQ ID NO: 23 - DNA, 20 bp, Artificial Sequence
  Description of Artificial Sequence Synthetic primer
tcaccgccttggcttgtcac

SEQ ID NO: 24 - DNA, 24 bp, Artificial Sequence
  Description of Artificial Sequence Synthetic primer
catcctgcaccaccaactgcttag

SEQ ID NO: 25 - DNA, 24 bp, Artificial Sequence
  Description of Artificial Sequence Synthetic primer
gcctgcttcaccaccttcttgatg

SEQ ID NO: 26 - DNA, 23 bp, Artificial Sequence
  Description of Artificial Sequence Synthetic primer
gccagcacatagagagaatgagc

SEQ ID NO: 27 - DNA, 23 bp, Artificial Sequence
  Description of Artificial Sequence Synthetic primer
caaggctcacagtgattttctgg

SEQ ID NO: 28 - protein, 10 aa, Artificial Sequence
  Description of Artificial Sequence Synthetic peptide
ACRGDMFGCA

SEQ ID NO: 29 - DNA, 21 bp, Artificial Sequence
  Description of Artificial Sequence Synthetic oligonucleotide
aagctgaccctgaagttcatc

SEQ ID NO: 30 - DNA, 21 bp, Artificial Sequence
  Description of Artificial Sequence Synthetic oligonucleotide
aagcagcacgacttcttcaag

SEQ ID NO: 31 - DNA, 21 bp, Homo sapiens
aagtggtcccaggctgcaccc

SEQ ID NO: 32 - DNA, 21 bp, Homo sapiens
aagatccgcagacgtgtaaat

SEQ ID NO: 33 - DNA, 21 bp, Homo sapiens
aaacacagactcgcgttgcaa

SEQ ID NO: 34 - DNA, 21 bp, Homo sapiens
aacacagactcgcgttgcaag

SEQ ID NO: 35 - DNA, 21 bp, Homo sapiens
aaggcgaggcagcttgagtta

SEQ ID NO: 36 - DNA, 21 bp, Homo sapiens
aaacgaacgtacttgcagatg

SEQ ID NO: 37 - DNA, 21 bp, Homo sapiens
aacgaacgtacttgcagatgt

SEQ ID NO: 38 - DNA, 21 bp, Homo sapiens
aacgtacttgcagatgtgaca

SEQ ID NO: 39 - DNA, 21 bp, Homo sapiens
aatcgagaccctggtggacat

SEQ ID NO: 40 - DNA, 21 bp, Homo sapiens
aaggccagcacataggagaga

SEQ ID NO: 41 - DNA, 21 bp, Homo sapiens
aaggaggagggcagaatcatc

SEQ ID NO: 42 - DNA, 21 bp, Homo sapiens
aatgcagaccaaagaaagata

SEQ ID NO: 43 - DNA, 21 bp, Homo sapiens
aatgtgaatgcagaccaaaga

SEQ ID NO: 44 - DNA, 21 bp, Homo sapiens
aacatcaccatgcagattatg

SEQ ID NO: 45 - DNA, 21 bp, Homo sapiens
aagcatttgtttgtacaagat

SEQ ID NO: 46 - DNA, 21 bp, Homo sapiens
aagtggtgaagttcatggatg

SEQ ID NO: 47 - DNA, 21 bp, Homo sapiens
aagatagagcaagacaagaaa

SEQ ID NO: 48 - DNA, 21 bp, Homo sapiens
aatccctgtgggccttgctca

SEQ ID NO: 49 - DNA, 21 bp, Homo sapiens
aaatgtgaatgcagaccaaag

SEQ ID NO: 50 - DNA, 21 bp, Homo sapiens
aatgacgagggcctggagtgt

SEQ ID NO: 51 - DNA, 21 bp, Homo sapiens
aaagtggtgtcatggatagat

SEQ ID NO: 52 - DNA, 21 bp, Homo sapiens
aagtggtgtcatggatagatg

SEQ ID NO: 53 - DNA, 21 bp, Homo sapiens
aaacagctggtgcccagctgc

SEQ ID NO: 54 - DNA, 21 bp, Homo sapiens
aagtccggatgcagatcctca

SEQ ID NO: 55 - DNA, 21 bp, Homo sapiens
aagaacacagccagtgtgaat

SEQ ID NO: 56 - DNA, 21 bp, Homo sapiens
aacacagccagtgtgaatgca

SEQ ID NO: 57 - DNA, 21 bp, Homo sapiens
aaaggacagtgctgtgaagcc

SEQ ID NO: 58 - DNA, 21 bp, Homo sapiens
aaggacagtgctgtgaagcca

SEQ ID NO: 59 - DNA, 21 bp, Homo sapiens
aagccagacagggctgccact

SEQ ID NO: 60 - DNA, 21 bp, Homo sapiens
aacccagacacctgcaggtgc

SEQ ID NO: 61 - DNA, 21 bp, Homo sapiens
aaggagaggacctgaaactgt

SEQ ID NO: 62 - DNA, 21 bp, Homo sapiens
aagcaaggagggcctctgatg

SEQ ID NO: 63 - DNA, 21 bp, Homo sapiens
aaggagggcctctgatggtga

SEQ ID NO: 64 - DNA, 21 bp, Homo sapiens
aactacctcaagagcaaacgt

SEQ ID NO: 65 - DNA, 21 bp, Homo sapiens
aagtggccagaggcatggagt

SEQ ID NO: 66 - DNA, 21 bp, Homo sapiens
aaagtgcattcatcgggacct

SEQ ID NO: 67 - DNA, 21 bp, Homo sapiens
aagtgcattcatcgggacctg

SEQ ID NO: 68 - DNA, 21 bp, Homo sapiens
agcacgctgtttattgaaaga

SEQ ID NO: 69 - DNA, 21 bp, Homo sapiens
aagggcttcatcatatcaaat

SEQ ID NO: 70 - DNA, 21 bp, Homo sapiens
aaaggctgagcataactaaat

SEQ ID NO: 71 - DNA, 21 bp, Homo sapiens
aaggtcttcttctgaaataaa

SEQ ID NO: 72 - DNA, 21 bp, Homo sapiens
aatgccatactgacaggaaat

SEQ ID NO: 73 - DNA, 21 bp, Homo sapiens
aagaggatgcagggaattata

SEQ ID NO: 74 - DNA, 21 bp, Homo sapiens
aaggcgacgaattgaccaaag

SEQ ID NO: 75 - DNA, 21 bp, Homo sapiens
aagatcctgaactgagtttaa

SEQ ID NO: 76 - DNA, 21 bp, Homo sapiens
aaggctgagcataactaaatc

SEQ ID NO: 77 - DNA, 21 bp, Homo sapiens
aaggccaagatttgcagaact

SEQ ID NO: 78 - DNA, 21 bp, Homo sapiens
aacacctcagtgcatatatat

SEQ ID NO: 79 - DNA, 21 bp, Homo sapiens
aaagggcttcatcatatcaaa

SEQ ID NO: 80 - DNA, 21 bp, Homo sapiens
aatcctccagaagaaagaaat

SEQ ID NO: 81 - DNA, 21 bp, Homo sapiens
aacagaatttcctgggacagc

SEQ ID NO: 82 - DNA, 21 bp, Homo sapiens
aactgaagacaggctacttgt

SEQ ID NO: 83 - DNA, 21 bp, Homo sapiens
aaggacttcctgaccttggag

SEQ ID NO: 84 - DNA, 21 bp, Homo sapiens
aagtggctaagggcatggagt

SEQ ID NO: 85 - DNA, 21 bp, Homo sapiens
aagggcatggagttcttggca

SEQ ID NO: 86 - DNA, 21 bp, Homo sapiens
aaatgtaccagaccatgctgg

SEQ ID NO: 87 - DNA, 21 bp, Homo sapiens
aattccattatgacaacacag

SEQ ID NO: 88 - DNA, 21 bp, Homo sapiens
aacagtaagcgaaagagccgg

SEQ ID NO: 89 - DNA, 21 bp, Homo sapiens
atgacaacacagcaggaatca

SEQ ID NO: 90 - DNA, 21 bp, Homo sapiens
aagctcagcacacagaaagac

SEQ ID NO: 91 - DNA, 21 bp, Homo sapiens
aatgcggcggtggtgacagta

SEQ ID NO: 92 - DNA, 21 bp, Homo sapiens
aatgccaccatgttctctaat

SEQ ID NO: 93 - DNA, 21 bp, Homo sapiens
aagctcctgaagatctgtata

SEQ ID NO: 94 - DNA, 21 bp, Homo sapiens
aagcagatgcctttggaattg

SEQ ID NO: 95 - DNA, 21 bp, Homo sapiens
aagcggctaccagtccggata

SEQ ID NO: 96 - DNA, 21 bp, Homo sapiens
aagtccctcagtgatgtagaa

SEQ ID NO: 97 - DNA, 21 bp, Homo sapiens
aagtgcttctaccgggaaact

SEQ ID NO: 98 - DNA, 21 bp, Homo sapiens
aatccctgtggatctgaaacg

SEQ ID NO: 99 - DNA, 21 bp, Homo sapiens
aaggctaatacaactcttcaa

SEQ ID NO: 100 - DNA, 21 bp, Homo sapiens
aatcccatttcaaaggagaag

SEQ ID NO: 101 - DNA, 21 bp, Homo sapiens
aagtggatagagagagctaat

SEQ ID NO: 102 - DNA, 21 bp, Homo sapiens
aaggctgctggattccagtat

SEQ ID NO: 103 - DNA, 21 bp, Homo sapiens
aagcagtctgtgattgaaatg

SEQ ID NO: 104 - DNA, 21 bp, Homo sapiens
aagccctcatcactggttgtg

SEQ ID NO: 105 - DNA, 21 bp, Homo sapiens
aaaggacatggttagaattaa

SEQ ID NO: 106 - DNA, 21 bp, Homo sapiens
aaggccttggccgtctggtta

SEQ ID NO: 107 - DNA, 21 bp, Homo sapiens
aagggtgtcaggtatttctta

SEQ ID NO: 108 - DNA, 21 bp, Homo sapiens
aatggagcgaagctttcatat

SEQ ID NO: 109 - DNA, 21 bp, Homo sapiens
aagtactgtgaagatgttaat

SEQ ID NO: 110 - DNA, 21 bp, Homo sapiens
aaggacatggttagaattaac

SEQ ID NO: 111 - DNA, 21 bp, Homo sapiens
aaggtactctcgcaggaaatg

SEQ ID NO: 112 - DNA, 21 bp, Homo sapiens
aaacggaggctgtgaacatat

SEQ ID NO: 113 - DNA, 21 bp, Homo sapiens
aatggccaagagattattctg

SEQ ID NO: 114 - DNA, 21 bp, Homo sapiens
aacctccattcatcatttgta

SEQ ID NO: 115 - DNA, 21 bp, Homo sapiens
aaggtctctcagttgaagaaa

SEQ ID NO: 116 - DNA, 21 bp, Homo sapiens
aatgccagctgcacaaataca

SEQ ID NO: 117 - DNA, 21 bp, Homo sapiens
aaggctctgttggagacatca

SEQ ID NO: 118 - DNA, 21 bp, Homo sapiens
aagaggactggcaaagataga

SEQ ID NO: 119 - DNA, 21 bp, Homo sapiens
aaggcaagagagagtatgtaa

SEQ ID NO: 120 - DNA, 21 bp, Homo sapiens
aagagagagtatgtaatatag

SEQ ID NO: 121 - DNA, 21 bp, Homo sapiens
aaagaccatccaggaggtggc

SEQ ID NO: 122 - DNA, 21 bp, Homo sapiens
aaagtgcctatcaagtggatg

SEQ ID NO: 123 - DNA, 21 bp, Homo sapiens
aagtgcctatcaagtggatgg

SEQ ID NO: 124 - DNA, 21 bp, Homo sapiens
aaccctgactaccagcaggac

SEQ ID NO: 125 - DNA, 21 bp, Homo sapiens
ctgactaccagcaggacttct

SEQ ID NO: 126 - DNA, 21 bp, Homo sapiens
aagatctttgggagcctggca

SEQ ID NO: 127 - DNA, 21 bp, Homo sapiens
aagaagatctttgggagcctg

SEQ ID NO: 128 - DNA, 21 bp, Homo sapiens
aaggtgcccatcaagtggatg

SEQ ID NO: 129 - DNA, 21 bp, Homo sapiens
aaatgttggatgattgactct

SEQ ID NO: 130 - DNA, 21 bp, Homo sapiens
aacctctattactgggaccag

SEQ ID NO: 131 - DNA, 21 bp, Homo sapiens
aattgactggagggacatcgt

SEQ ID NO: 132 - DNA, 21 bp, Homo sapiens
aagatcctgggcaacctggac

SEQ ID NO: 133 - DNA, 21 bp, Homo sapiens
aaggaaattagtgctgggcgt

SEQ ID NO: 134 - DNA, 21 bp, Homo sapiens
aagattccagtctgcattaaa

SEQ ID NO: 135 - DNA, 21 bp, Homo sapiens
aaatacacacaccagagtgat

SEQ ID NO: 136 - DNA, 21 bp, Homo sapiens
aatacacacaccagagtgatg

SEQ ID NO: 137 - DNA, 21 bp, Homo sapiens
aagatgaagatgaggagtatg

SEQ ID NO: 138 - DNA, 21 bp, Homo sapiens
aacctctattactgggaccag

SEQ ID NO: 139 - DNA, 21 bp, Homo sapiens
ctgacaagatggaagtagata

SEQ ID NO: 140 - DNA, 21 bp, Homo sapiens
tgacaagatggaagtagataa

SEQ ID NO: 141 - DNA, 21 bp, Homo sapiens
tcaagattccagtctgcatta

SEQ ID NO: 142 - DNA, 21 bp, Homo sapiens
caagattccagtctgcattaa

SEQ ID NO: 143 - DNA, 21 bp, Homo sapiens
tgaggccaagactccaattaa

SEQ ID NO: 144 - DNA, 21 bp, Homo sapiens
aaatggtggagtctatgtaga

SEQ ID NO: 145 - DNA, 21 bp, Homo sapiens
aatggtggagtctatgtagac

SEQ ID NO: 146 - DNA, 21 bp, Homo sapiens
aatgtgctggaggctgctcag

SEQ ID NO: 147 - DNA, 21 bp, Homo sapiens
aatccaaccacctttcaactg

SEQ ID NO: 148 - DNA, 21 bp, Homo sapiens
aacaggtttcctgaacataca

SEQ ID NO: 149 - DNA, 21 bp, Homo sapiens
aactggacaacactcttcagc

SEQ ID NO: 150 - DNA, 21 bp, Homo sapiens
aacggtcccactagtcatgac

SEQ ID NO: 151 - DNA, 21 bp, Homo sapiens
aagagcgaccctcacatcaag

SEQ ID NO: 152 - DNA, 21 bp, Homo sapiens
aagcagaagagagaggagttg

SEQ ID NO: 153 - DNA, 21 bp, Homo sapiens
aaacgaactgggcagtataaa

SEQ ID NO: 154 - DNA, 21 bp, Homo sapiens
aaacaggacctgggcagaaag

SEQ ID NO: 155 - DNA, 21 bp, Homo sapiens
aactgggcagtataaacttgg

SEQ ID NO: 156 - DNA, 21 bp, Homo sapiens
aagctacaacttcaagcagaa

SEQ ID NO: 157 - DNA, 21 bp, Homo sapiens
aacgaactgggcagtataaac

SEQ ID NO: 158 - DNA, 21 bp, Homo sapiens
aacaggacctgggcagaaagc

SEQ ID NO: 159 - DNA, 21 bp, Homo sapiens
aagagagaggagttgtgtcta

SEQ ID NO: 160 - DNA, 21 bp, Homo sapiens
aaggaagatggaagattactg

SEQ ID NO: 161 - DNA, 21 bp, Homo sapiens
aagattactggcttctaaatg

SEQ ID NO: 162 - DNA, 21 bp, Homo sapiens
aacgattggaatctaataact

SEQ ID NO: 163 - DNA, 21 bp, Homo sapiens
aaaggagtgtgtgctaaccgt

SEQ ID NO: 164 - DNA, 21 bp, Homo sapiens
aaggaaatacaccagttggta

SEQ ID NO: 165 - DNA, 21 bp, Homo sapiens
aatacttaccggtcaaggaaa

SEQ ID NO: 166 - DNA, 21 bp, Homo sapiens
aaatgtgttacggatgagtgt

SEQ ID NO: 167 - DNA, 21 bp, Homo sapiens
aaatacaccagttggtatgtg

SEQ ID NO: 168 - DNA, 21 bp, Homo sapiens
aacttcaagcagaagagagag

SEQ ID NO: 169 - DNA, 21 bp, Homo sapiens
aatacaccagttggtatgtgg

SEQ ID NO: 170 - DNA, 21 bp, Homo sapiens
aactacaatacttaccggtca

SEQ ID NO: 171 - DNA, 21 bp, Homo sapiens
aaggctggaggagaaccatta

SEQ ID NO: 172 - DNA, 21 bp, Homo sapiens
aagcccaaactcctctactgt

SEQ ID NO: 173 - DNA, 21 bp, Homo sapiens
aatctgcctccagggaattac

SEQ ID NO: 174 - DNA, 21 bp, Homo sapiens
aagcgccacaagcagcagctg

SEQ ID NO: 175 - DNA, 21 bp, Homo sapiens
aagaagcatgcagagaagaat

SEQ ID NO: 176 - DNA, 21 bp, Homo sapiens
aacgcggtcctcggactcact

SEQ ID NO: 177 - DNA, 21 bp, Homo sapiens
aaccattacaacacctatata

SEQ ID NO: 178 - DNA, 21 bp, Homo sapiens
aagctctttagtcttgaaagc

SEQ ID NO: 179 - DNA, 21 bp, Homo sapiens
aacacctatatatccaagaag

SEQ ID NO: 180 - DNA, 21 bp, Homo sapiens
aactcctctactgtagcaacg

SEQ ID NO: 181 - DNA, 21 bp, Homo sapiens
aagggacaggagcgaccagca

SEQ ID NO: 182 - DNA, 21 bp, Homo sapiens
aagcatgcagagaagaattgg

SEQ ID NO: 183 - DNA, 21 bp, Homo sapiens
aaagcgccacaagcagcagct

SEQ ID NO: 184 - DNA, 21 bp, Homo sapiens
aattggtttgttggcctcaag

SEQ ID NO: 185 - DNA, 21 bp, Homo sapiens
aagaagaatgggagctgcaaa

SEQ ID NO: 186 - DNA, 21 bp, Homo sapiens
aagaagcccaaactcctctac

SEQ ID NO: 187 - DNA, 21 bp, Homo sapiens
aaacgcggtcctcggactcac

SEQ ID NO: 188 - DNA, 21 bp, Homo sapiens
aatgggagctgcaaacgcggt

SEQ ID NO: 189 - DNA, 21 bp, Homo sapiens
aaactcctctactgtagcaac

SEQ ID NO: 190 - DNA, 21 bp, Homo sapiens
aatgaggaatgtttgttcctg

SEQ ID NO: 191 - DNA, 21 bp, Homo sapiens
aagccggactgccggcaaatg

SEQ ID NO: 192 - DNA, 21 bp, Homo sapiens
aagccctgtttgatagagtat

SEQ ID NO: 193 - DNA, 21 bp, Homo sapiens
aagcagtgggaattgacaaag

SEQ ID NO: 194 - DNA, 21 bp, Homo sapiens
aaaggcaacctccgagaatac

SEQ ID NO: 195 - DNA, 21 bp, Homo sapiens
aatcgcctgtatggtggtaac

SEQ ID NO: 196 - DNA, 21 bp, Homo sapiens
aatgggagtttccaagagata

SEQ ID NO: 197 - DNA, 21 bp, Homo sapiens
aagagccaccaaccaaatacc

SEQ ID NO: 198 - DNA, 21 bp, Homo sapiens
aagcagttggtagaagacttg

SEQ ID NO: 199 - DNA, 21 bp, Homo sapiens
aagcaggagcatcgcattgga

SEQ ID NO: 200 - DNA, 21 bp, Homo sapiens
aagcggctccatgctgtgcct

SEQ ID NO: 201 - DNA, 21 bp, Homo sapiens
aagagattgaggttctctata

SEQ ID NO: 202 - DNA, 21 bp, Homo sapiens
aacagtcatcctgtgccgaat

SEQ ID NO: 203 - DNA, 21 bp, Homo sapiens
aagccagccaactgcaccaac

SEQ ID NO: 204 - DNA, 21 bp, Homo sapiens
aaggagtttaagcaggagcat

SEQ ID NO: 205 - DNA, 21 bp, Homo sapiens
aagatgatgccacagagaaag

SEQ ID NO: 206 - DNA, 21 bp, Homo sapiens
aaggacacagaatggataagc

SEQ ID NO: 207 - DNA, 21 bp, Homo sapiens
aaacgggaaggagtttaagca

SEQ ID NO: 208 - DNA, 21 bp, Homo sapiens
aaaccagcactggagcctcat

SEQ ID NO: 209 - DNA, 21 bp, Homo sapiens
aagctgctgaaggaaggacac

SEQ ID NO: 210 - DNA, 21 bp, Homo sapiens
aaagagattgaggttctctat

SEQ ID NO: 211 - DNA, 21 bp, Homo sapiens
aacggccgactgcctgtgaag

SEQ ID NO: 212 - DNA, 21 bp, Homo sapiens
aagtcggacgcaacagagaaa

SEQ ID NO: 213 - DNA, 21 bp, Homo sapiens
aagggcaacctgcgggagtac

SEQ ID NO: 214 - DNA, 21 bp, Homo sapiens
aagtggctgtgaagatgttga

SEQ ID NO: 215 - DNA, 21 bp, Homo sapiens
aatggagatgatgaagatgat

SEQ ID NO: 216 - DNA, 21 bp, Homo sapiens
aacccaaccgtgtgaccaaag

SEQ ID NO: 217 - DNA, 21 bp, Homo sapiens
aagcccagtaactgcaccaac

SEQ ID NO: 218 - DNA, 21 bp, Homo sapiens
aacgtggagttcatgtgtaag

SEQ ID NO: 219 - DNA, 21 bp, Homo sapiens
aaacccaaccgtgtgaccaaa

SEQ ID NO: 220 - DNA, 21 bp, Homo sapiens
aagatgatcgggaagcataag

SEQ ID NO: 221 - DNA, 21 bp, Homo sapiens
aacaccaaaccaaaccgtatg

SEQ ID NO: 222 - DNA, 21 bp, Homo sapiens
aatggcaaagaattcaaacct

SEQ ID NO: 223 - DNA, 21 bp, Homo sapiens
aacgagctgtacatgatgatg

SEQ ID NO: 224 - DNA, 21 bp, Homo sapiens
aacctgccttatgtccagatc

SEQ ID NO: 225 - DNA, 21 bp, Homo sapiens
aagctgctgaaggagggtcac

SEQ ID NO: 226 - DNA, 21 bp, Homo sapiens
aaagcacatcgaggtgaatgg

SEQ ID NO: 227 - DNA, 21 bp, Homo sapiens
aaggacaaacccaaccgtgtg

SEQ ID NO: 228 - DNA, 21 bp, Homo sapiens
aagcagctggtggaagacctg

SEQ ID NO: 229 - DNA, 21 bp, Homo sapiens
aaagtggctgtgaagatgttg

SEQ ID NO: 230 - DNA, 21 bp, Homo sapiens
aaccacacataccagctggat

SEQ ID NO: 231 - DNA, 21 bp, Homo sapiens
aacctcgactactacaagaag

SEQ ID NO: 232 - DNA, 21 bp, Homo sapiens
aagatgatcgggaaacacaaa

SEQ ID NO: 233 - DNA, 21 bp, Homo sapiens
aaggacctgtcggacctggtg

SEQ ID NO: 234 - DNA, 21 bp, Homo sapiens
aaggtgtacagtgacgcacag

SEQ ID NO: 235 - DNA, 21 bp, Homo sapiens
aagcagctggtggaggacctg

SEQ ID NO: 236 - DNA, 21 bp, Homo sapiens
aagctgcggcatcagcagtgg

SEQ ID NO: 237 - DNA, 21 bp, Homo sapiens
aagcctgtcaccgtagccgtg

SEQ ID NO: 238 - DNA, 21 bp, Homo sapiens
aagacgatgccactgacaagg

SEQ ID NO: 239 - DNA, 21 bp, Homo sapiens
aacgcgtccatgagctccaac

SEQ ID NO: 240 - DNA, 21 bp, Homo sapiens
aagcgacaggtgtccctggag

SEQ ID NO: 241 - DNA, 21 bp, Homo sapiens
aactgcacacacgacctgtac

SEQ ID NO: 242 - DNA, 21 bp, Homo sapiens
aaggagctagaggttctctcc

SEQ ID NO: 243 - DNA, 21 bp, Homo sapiens
aaagacgatgccactgacaag

SEQ ID NO: 244 - DNA, 21 bp, Homo sapiens
aacaccaccgacaaggagcta

SEQ ID NO: 245 - DNA, 21 bp, Homo sapiens
aagtgcatccacagggacctg

SEQ ID NO: 246 - DNA, 21 bp, Homo sapiens
aatgcctcccacgaggactcc

SEQ ID NO: 247 - DNA, 21 bp, Homo sapiens
aaggacctggtgtcctgtgcc

SEQ ID NO: 248 - DNA, 21 bp, Homo sapiens
aagctgctgaaggagggccac

SEQ ID NO: 249 - DNA, 21 bp, Homo sapiens
aacctgcgggagtttctgcgg

SEQ ID NO: 250 - DNA, 21 bp, Homo sapiens
aaggatggcacagggctggtg

SEQ ID NO: 251 - DNA, 21 bp, Homo sapiens
aaggatggacaggcctttcat

SEQ ID NO: 252 - DNA, 21 bp, Homo sapiens
aaggtcctgctggccgtctct

SEQ ID NO: 253 - DNA, 21 bp, Homo sapiens
aagctgatcggccgacacaag

SEQ ID NO: 254 - DNA, 21 bp, Homo sapiens
aaagactgcagacatcaatag

SEQ ID NO: 255 - DNA, 21 bp, Homo sapiens
aagagcaggagctgacagtag

SEQ ID NO: 256 - DNA, 21 bp, Homo sapiens
aagccagcactgtggccgtca

SEQ ID NO: 257 - DNA, 21 bp, Homo sapiens
aaccgcattggaggcattcgg

SEQ ID NO: 258 - DNA, 21 bp, Homo sapiens
aagggaaacctgcgggagttc

SEQ ID NO: 259 - DNA, 21 bp, Homo sapiens
aagctctcccgcttccctctg

SEQ ID NO: 260 - DNA, 21 bp, Homo sapiens
aagtcctaaagactgcagaca

SEQ ID NO: 261 - DNA, 21 bp, Homo sapiens
aacgcctctgacaaggacctg

SEQ ID NO: 262 - DNA, 21 bp, Homo sapiens
aagcaccctactggacacacc

SEQ ID NO: 263 - DNA, 21 bp, Homo sapiens
aagactgcagacatcaatagc

SEQ ID NO: 264 - DNA, 21 bp, Homo sapiens
aaagacaacgcctctgacaag

SEQ ID NO: 265 - DNA, 21 bp, Homo sapiens
aacaccgtcaagttccgctgt

SEQ ID NO: 266 - DNA, 21 bp, Homo sapiens
aagctcatccctggtacgagg

SEQ ID NO: 267 - DNA, 21 bp, Homo sapiens
aaggtgtacagcgatgcccag

SEQ ID NO: 268 - DNA, 21 bp, Homo sapiens
aagacaacgcctctgacaagg

SEQ ID NO: 269 - DNA, 21 bp, Homo sapiens
aacatcatcaacctgcttggt

SEQ ID NO: 270 - DNA, 21 bp, Homo sapiens
aatctcaccttgattacaggt

SEQ ID NO: 271 - DNA, 21 bp, Homo sapiens
aagtcaaccacagagtcgtat

SEQ ID NO: 272 - DNA, 21 bp, Homo sapiens
aagtaacgagtgagccacgct

SEQ ID NO: 273 - DNA, 21 bp, Homo sapiens
aagtcgagtgtgctactcaac

SEQ ID NO: 274 - DNA, 21 bp, Homo sapiens
aactgaacttccggcagaaac

SEQ ID NO: 275 - DNA, 21 bp, Homo sapiens
aatgcggagaacactaattat

SEQ ID NO: 276 - DNA, 21 bp, Homo sapiens
aacttccataaatgtgaaatc

SEQ ID NO: 277 - DNA, 21 bp, Homo sapiens
aagtgatactcccgcctcagc

SEQ ID NO: 278 - DNA, 21 bp, Homo sapiens
aagtagctggcactacgggca

SEQ ID NO: 279 - DNA, 21 bp, Homo sapiens
aatcaggttccaatgtgatga

SEQ ID NO: 280 - DNA, 21 bp, Homo sapiens
aaggcttagctcccaagcctc

SEQ ID NO: 281 - DNA, 21 bp, Homo sapiens
aaggcagatccagctgtggca

SEQ ID NO: 282 - DNA, 21 bp, Homo sapiens
aagccagagtcgtcccctggc

SEQ ID NO: 283 - DNA, 21 bp, Homo sapiens
aagtcttccgttttctgagaa

SEQ ID NO: 284 - DNA, 21 bp, Homo sapiens
aatggtgcagcagaaattgga

SEQ ID NO: 285 - DNA, 21 bp, Homo sapiens
aagaagggccaccagctgctg

SEQ ID NO: 286 - DNA, 21 bp, Homo sapiens
aacgtcaccgacggcggccac

SEQ ID NO: 287 - DNA, 21 bp, Homo sapiens
aacctcgggcagaagaggaga

SEQ ID NO: 288 - DNA, 21 bp, Homo sapiens
aactgaaacggattgccagag

SEQ ID NO: 289 - DNA, 21 bp, Homo sapiens
aagaagcgatacaggtctcgt

SEQ ID NO: 290 - DNA, 21 bp, Homo sapiens
aaggtctcgtagtagagatcg

SEQ ID NO: 291 - DNA, 21 bp, Homo sapiens
aacctggatcaggtccaagca

SEQ ID NO: 292 - DNA, 21 bp, Homo sapiens
aatctgaagtcatgcttggac

SEQ ID NO: 293 - DNA, 21 bp, Homo sapiens
aacagaggaggactacattcc

SEQ ID NO: 294 - DNA, 21 bp, Homo sapiens
aaccacgaaatcaccagcatc

SEQ ID NO: 295 - DNA, 21 bp, Homo sapiens
aagctggacattccctctgcg

SEQ ID NO: 296 - DNA, 21 bp, Homo sapiens
aagagcccagcttcctgcagc

SEQ ID NO: 297 - DNA, 21 bp, Homo sapiens
aactgttgaggagcccatgga

SEQ ID NO: 298 - DNA, 21 bp, Homo sapiens
aatctgatgatgaagctgcag

SEQ ID NO: 299 - DNA, 21 bp, Homo sapiens
aaccgcggtcctattccatta

SEQ ID NO: 300 - DNA, 21 bp, Homo sapiens
aacactccaatttttcaaagt

SEQ ID NO: 301 - DNA, 21 bp, Homo sapiens
aagagttaccttcctaatgca

SEQ ID NO: 302 - DNA, 21 bp, Homo sapiens
aagattgggttggtatattca

SEQ ID NO: 303 - DNA, 21 bp, Homo sapiens
aatccttgttctcactgagct

SEQ ID NO: 304 - DNA, 21 bp, Homo sapiens
aagatggctgagctggggctg

SEQ ID NO: 305 - DNA, 21 bp, Homo sapiens
aacccctgccacaacggtggt

SEQ ID NO: 306 - DNA, 21 bp, Homo sapiens
aaccactgtgagacgaaatgt

SEQ ID NO: 307 - DNA, 21 bp, Homo sapiens
aactgccccagcgatctctgc

SEQ ID NO: 308 - DNA, 21 bp, Homo sapiens
aacctaattctcctgaggctg

SEQ ID NO: 309 - DNA, 21 bp, Homo sapiens
aagagtgacactgtggcttcc

SEQ ID NO: 310 - DNA, 21 bp, Homo sapiens
aatgggctgagctgctgctcc

SEQ ID NO: 311 - DNA, 21 bp, Homo sapiens
aagcctgtagcccatgttgta

SEQ ID NO: 312 - DNA, 21 bp, Homo sapiens
aatggcgtggagctgagagat

SEQ ID NO: 313 - DNA, 21 bp, Homo sapiens
aacctcctctctgccatcaag

SEQ ID NO: 314 - DNA, 21 bp, Homo sapiens
aaccagctggtggtgccatca

SEQ ID NO: 315 - DNA, 21 bp, Homo sapiens
aagccctggtatgagcccatc

SEQ ID NO: 316 - DNA, 21 bp, Homo sapiens
aatgccctcctggccaatggc

SEQ ID NO: 317 - DNA, 21 bp, Homo sapiens
aagggtgaccgactcagcgct

SEQ ID NO: 318 - DNA, 21 bp, Homo sapiens
aagcatgatccgggacgtgga

SEQ ID NO: 319 - DNA, 21 bp, Homo sapiens
aaggtcaacctcctctctgcc

SEQ ID NO: 320 - DNA, 21 bp, Homo sapiens
aaagcatgatccgggacgtgg

SEQ ID NO: 321 - DNA, 21 bp, Homo sapiens
aagaaccagtaccggcattat

SEQ ID NO: 322 - DNA, 21 bp, Homo sapiens
aagctctactccattgtttgt

SEQ ID NO: 323 - DNA, 21 bp, Homo sapiens
aagccacagagcctagacact

SEQ ID NO: 324 - DNA, 21 bp, Homo sapiens
aaagcctggagtgcacgaagt

SEQ ID NO: 325 - DNA, 21 bp, Homo sapiens
aaggaacctacttgtacaatg

SEQ ID NO: 326 - DNA, 21 bp, Homo sapiens
aattgcagcctctgcctcaat

SEQ ID NO: 327 - DNA, 21 bp, Homo sapiens
aatgggtcaggtggagatctc

SEQ ID NO: 328 - DNA, 21 bp, Homo sapiens
aaccagtaccggcattattgg

SEQ ID NO: 329 - DNA, 21 bp, Homo sapiens
aaaggaacctacttgtacaat

SEQ ID NO: 330 - DNA, 21 bp, Homo sapiens
aagtgccacaaaggaacctac

SEQ ID NO: 331 - DNA, 21 bp, Homo sapiens
aaatgggtcaggtggagatct

SEQ ID NO: 332 - DNA, 21 bp, Homo sapiens
aacgagtgtgtctcctgtagt

SEQ ID NO: 333 - DNA, 21 bp, Homo sapiens
aagggcactgaggactcaggc

SEQ ID NO: 334 - DNA, 21 bp, Homo sapiens
aaacgagtgtgtctcctgtag

SEQ ID NO: 335 - DNA, 21 bp, Homo sapiens
aacggtggaagtccaagctct

SEQ ID NO: 336 - DNA, 21 bp, Homo sapiens
aacaccgtgtgcacctgccat

SEQ ID NO: 337 - DNA, 21 bp, Homo sapiens
aatgggaccgtgcacctctcc

SEQ ID NO: 338 - DNA, 21 bp, Homo sapiens
aacccaagcttcagtcccact

SEQ ID NO: 339 - DNA, 21 bp, Homo sapiens
aacctacttgtacaatgactg

SEQ ID NO: 340 - DNA, 21 bp, Homo sapiens
aattcgatttgctgtaccaag

SEQ ID NO: 341 - DNA, 21 bp, Homo sapiens
aagggagcactggcgacttcg

SEQ ID NO: 342 - DNA, 21 bp, Homo sapiens
aagcccttgtgcctgcagaga

SEQ ID NO: 343 - DNA, 21 bp, Homo sapiens
aagcctgcactcgggaacaga

SEQ ID NO: 344 - DNA, 21 bp, Homo sapiens
aaggaggaatgtgcctttcgg

SEQ ID NO: 345 - DNA, 21 bp, Homo sapiens
aagacctcggacaccgtgtgt

SEQ ID NO: 346 - DNA, 21 bp, Homo sapiens
aactgggttcccgagtgcttg

SEQ ID NO: 347 - DNA, 21 bp, Homo sapiens
aacccagcactgctccaagca

SEQ ID NO: 348 - DNA, 21 bp, Homo sapiens
aatgggagacacagattccag

SEQ ID NO: 349 - DNA, 21 bp, Homo sapiens
aagccaaggtgcctcacttgc

SEQ ID NO: 350 - DNA, 21 bp, Homo sapiens
aataggagtggtgaactgtgt

SEQ ID NO: 351 - DNA, 21 bp, Homo sapiens
aatgtcacctgcatcgtgaac

SEQ ID NO: 352 - DNA, 21 bp, Homo sapiens
aacacgacttcatccacggat

SEQ ID NO: 353 - DNA, 21 bp, Homo sapiens
aagccagctccacaatgggag

SEQ ID NO: 354 - DNA, 21 bp, Homo sapiens
aaccgcatctgcacctgcagg

SEQ ID NO: 355 - DNA, 21 bp, Homo sapiens
aaggtgcctcacttgcctgcc

SEQ ID NO: 356 - DNA, 21 bp, Homo sapiens
aagcacctccttcctgctccc

SEQ ID NO: 357 - DNA, 21 bp, Homo sapiens
aagaagcccttgtgcctgcag

SEQ ID NO: 358 - DNA, 21 bp, Homo sapiens
aacgtctgtagcagctctgac

SEQ ID NO: 359 - DNA, 21 bp, Homo sapiens
aatgtgcctttcggtcacagc

SEQ ID NO: 360 - DNA, 21 bp, Homo sapiens
aactccagaacccagcactgc

SEQ ID NO: 361 - DNA, 21 bp, Mus musculus
aggctccgagatgaacaacaa

SEQ ID NO: 362 - DNA, 21 bp, Mus musculus
tacctgtcctgtgtaatgaaa

SEQ ID NO: 363 - DNA, 21 bp, Mus musculus
accatcgaggttactaatgaa

SEQ ID NO: 364 - DNA, 21 bp, Mus musculus
tcggaatatctcccatcataa

SEQ ID NO: 365 - DNA, 21 bp, Mus musculus
tcgggaggagacgactctaaa

SEQ ID NO: 366 - DNA, 21 bp, Mus musculus
ccagagtgatttgagatacaa

SEQ ID NO: 367 - DNA, 21 bp, Mus musculus
cacgtttatctcggctgctta

SEQ ID NO: 368 - DNA, 21 bp, Mus musculus
aagactgatagtcccgtgcaa

SEQ ID NO: 369 - DNA, 21 bp, Mus musculus
aaggaaagtatgtccattcta

SEQ ID NO: 370 - DNA, 21 bp, Mus musculus
ccgcaacgtcctgacaatgca

SEQ ID NO: 371 - DNA, 21 bp, Mus musculus
ccaggttgtcttgacacccta

SEQ ID NO: 372 - DNA, 21 bp, Mus musculus
ctggctattcccggaaatgca

SEQ ID NO: 373 - DNA, 21 bp, Mus musculus
cacgtcgtagcaaaccaccaa

SEQ ID NO: 374 - DNA, 21 bp, Mus musculus
cagccgatttgctatctcata

(19) United States
(12) Patent Application Publication
(10) Pub. No.: US 2009/0247604 A1
(43) Pub. Date: Oct. 1, 2009

(54) RNAi Therapeutics for Treatment of Eye Neovascularization Diseases

(75) Inventors: Quinn Tang, Gaithersburg, MD (US); Patrick Y. Lu, Rockville, MD (US); Frank Y. Xie, Germantown, MD (US); Martin C. Woodle, Bethesda, MD (US)

Correspondence Address: ROPES & GRAY LLP, PATENT DOCKETING 39/361, 1211 AVENUE OF THE AMERICAS, NEW YORK, NY, 10036-8704, US

(73) Assignee: Intradigm Corporation, Rockville, MD (US)

(21) Appl. No.: 10/588,602

(22) PCT Filed: Feb. 7, 2005
(86) PCT No.: PCT/US05/03857

§ 371 Date: Jun. 13, 2007

Related U.S. Application Data
(60) Provisional application No. 60/541,775, filed on Feb. 5, 2004.

(51) Int. Cl. A61K 31/713; A61P 27/02

(52) U.S. Cl. 514/44 A

(57) ABSTRACT

Compositions and methods for treating ocular disease are provided. Specifically, siRNA molecules and mixtures of siRNA molecules are provided that inhibit angiogenesis and/or neovascularization. The compositions and methods are suitable for treating ocular diseases associated with angiogenesis and/or neovascularization.

This application claims priority to U.S. Provisional application Ser. No. 60/541,775, filed Feb. 5, 2004, the contents of which are hereby incorporated by reference in their entirety.

### BACKGROUND

Many diverse ocular diseases are the result of excessive neovascularization (NV), an abnormal proliferation and growth of blood vessels within the eye. The development of ocular NV itself has adverse consequences for vision but also is an early pathological step in many serious eye diseases; despite introduction of new therapeutic agents it remains the most common cause of permanent blindness in United States and Europe. Several major eye diseases promote an abnormal neovascularization, which leads to further damage to the eyes causing loss of vision. Unfortunately, few treatment options exist for patients with any of these ocular NV diseases. The most commonly used approved therapy is a photodynamic treatment, Visudyne, that uses light to activate a photosensitizer in the vicinity of the neovascularization to destroy unwanted blood vessels. It is not effective in many patients and cannot prevent recurrence even when it is effective. A recently approved agent, Macugen, provides some benefit but also is ineffective in most patients. In addition, the intraocular administration of Macugen leads to irritation and risk of infection, both of which are adverse since they exacerbate the neovascularization pathology. As a consequence, a large and growing unmet clinical need exists for effective treatments, either inhibiting progression since disease progression tends to be very prolonged or therapeutic to reverse the unwanted angiogenesis.

The National Eye Institute of NIH has estimated, 400,000 Americans have had some form of ocular herpes, and there are nearly 50,000 new and recurring cases diagnosed each year in the United States, with the more serious stromal keraitits accounting for about 25%. From a larger study, it was found that the recurrence rate of ocular herpes is 10 percent in one year, 23 percent in two years, and 63 percent within 20 years. Although application of available anti-viral drugs could control the HSV infection to certain extent, there is no effective medication available that could treat the HSV-caused stromal keratitis and protect the patients from blindness.

The ocular neovascularization diseases can be divided into diseases affecting the anterior, or front, of the eye and those affecting the posterior, or retinal, part of the eye. Development of NV at these different regions may have different origins, but the biochemical and physiological nature of the NV process appears to be virtually identical, regardless of eye region. Consequently, an effective means to intervene in the biochemical nature of ocular NV offers the prospect for providing an effective treatment for any ocular disease that involves ocular NV as the major pathology or as the underlying pathology, regardless of whether the disease afflicts the anterior or posterior of the eye. Nonetheless, the anterior and posterior ocular tissues differ considerably and these differences can have a dramatic influence on the most effective means to administer therapeutic treatments so that the tissue and cells are reached by the therapeutic agent.

Posterior Ocular NV Disease

Diabetic Retinopathy (DR) occurs when the tiny blood vessels providing oxygen to the retina become damaged. The damage allows blood and fluid to escape into the retina, and also results in new blood vessel growth. These new vessels are more fragile and frequently bleed into the vitreous region of the eye, interfering in vision. Patients with the most serious form of DR are at a substantial risk for severe visual loss without treatment. In this disease, neovascularization is a central pathology of the disease.

Age related macular degeneration (AMD) is the leading cause of blindness in people over 60 years and each year the problem becomes more acute. In AMD central vision is lost making it impossible to appreciate fine detail. Given the magnitude of the burden of AMD on individuals and society as a whole, it is perhaps surprising that more is not known of the causes of the disease and how it develops. It is clear, however, that the retinal pigment epithelium (RPE) plays a pivotal role. Abnormal waste material builds up beneath and within the RPE and RPE cells eventually die. The rods and cones in the retina depend for their survival upon normal functioning RPE and this RPE failure leads to progressive loss of vision. The disease provokes a scarring process at the back of the eye inducing formation of new blood vessels, neovascularization. In a large segment of the patients, those with “wet AMD”, an excessive proliferation of leaky neovasculature develops in front of the retina, which also blurs and distorts vision. In this disease, damaging neovascularization develops in later, severe stages of disease in a large segment of the patient population.

Uveitis is an eye disease originating from excessive or persistent inflammation of the tissue on the inside of the eye. Over time uveitis leads to neovascularization that damages vision. This disease can develop in several different regions of the eye such as localized in the posterior or diffuse throughout many regions including the posterior region. This disease originates from inflammation, which can arise from a wide variety of causes including viral infections. The commonality is the excessive and persistent inflammation leading to damaging pathological processes, including neovascularization, and ultimately loss of vision.

Anterior Ocular NV Disease

Rubeosis is a term that describes abnormal blood vessel growth on the iris and the structures in the front of the eye. Normally there are no visible blood vessels in these areas. When the retina has been deprived of oxygen, or is ischemic, as with diabetic retinopathy or vein occlusion, abnormal vessels form to supply oxygen to the eye. Unfortunately, the formation of these vessels obstructs the drainage of aqueous fluid from the front of the eye, causing the eye pressure to become elevated. This usually leads to neovascular glaucoma.

Uveitis is a broad group of diseases originating from inflammation of tissues on the inside of the eye. This disease is most commonly classified anatomically as anterior, intermediate, posterior or diffuse. Ocular complications of uveitis may produce profound and irreversible loss of vision, especially when unrecognized or treated improperly. The most frequent complications include cataract; glaucoma; retinal detachment; neovascularization of the retina, optic nerve, or iris and the like.

Choroidal Neovascularization (CNV) is the pathology underlying a broad group of ocular diseases, characterized by neovascularization in this region of the eye. A related group of ocular diseases are the consequence of eye infections, including Conjunctivitis, Keratitis, Blepharitis, Sty, Chalazion and Iritis, again all major causes of ocular neovascularization that leads to vision loss. Recurrent HSV infection is the most common infectious cause of corneal blindness in the U.S. This viral infection causes blinding lesions called stromal keratitis (SK). Corneal NV is an early step in vision loss from herpetic SK.

Ocular NV Biochemistry and Physiology

Like other tissues, ocular tissues are in a continuous state of maintenance which often entails neovascularization. This essential process is kept in balance by a balance of pro- and inhibitory factors. Unfortunately, the balance is not correctly maintained in the many ocular neovascularization diseases and excessive growth of damaging new blood vessels are the result. The process for this excessive neovascularization appears to be virtually identical regardless of the region of the eye and disease, although the originating cause of the pathology as well as the role in vision loss differs widely. The commonality of the pathological process offers means to provide therapeutic interventions that are effective in these diverse diseases of the eye.

The normal cornea is avascular and HSV does not express any angiogenic protein itself, but infected ocular tissues express angiogenic factors that induce corneal NV. The angiogenic factor production occurs initially from virus-infected corneal epithelial, non-inflammatory, cells followed by expression in a clinical phase from inflammatory cells (PMNs and macrophages) in the stroma. A mouse model of HSV induced corneal NV was developed by implantation of purified HSV viral DNA fragments (HSV DNA, rich in CpG motifs) or synthetic CpG oligonucleotides (CpG ODN). This model is thought to provide a clinically relevant model of corneal NV and herpetic SK disease, and is useful for testing therapeutic modalities for their efficacy in inhibiting ocular NV disease.

An attractive approach for therapeutic intervention is to inhibit the common pathological condition of these diseases. From many studies, it has become established that VEGF-mediated neovascularization and angiogenesis is one of the common pathological pathways of many ocular neovascularization diseases. The VEGF-mediated angiogenesis pathway plays a central role in angiogenesis of all these NV-related eye diseases. The VEGF family is composed of five structurally related growth factors: VEGF-A, Placenta Growth factor (PIGF), VEGF-B, VEGF-C, and VEGF-D. Known receptors include three structurally homologous tyrosine kinase receptors, VEGFR-1 (Flt-1), VEGFR-2 (KDR or Flk-1), and VEGFR-3 (Flt4), with different affinity or functions related to different VEGF members. While function and regulation of four VEGF members are poorly understood, VEGF-A binds VEGFR-1 and VEGFR-2 and is known to induce neovascularization and angiogenesis, as well as vascular permeability. VEGFR-1 and VEGFR-2 are both up-regulated in proliferating endothelium that may be a direct response to VEGF-A or hypoxia. VEGFR-1 has higher affinity to VEGF-A than VEGFR-2. It is thought that VEGFR-2 is responsible for angiogenic signals for blood vessel growth, but the function of VEGFR-1 is poorly understood. Some studies suggested a direct role in transducing angiogenic signals, and roles in motility and permeability. This understanding of key players in the VEGF pathway of angiogenesis has led to studies with inhibitors of VEGF-A as candidate therapeutic agents, including macugen, an aptamer oligonucleotide inhibiting VEGF binding to its receptor. While these studies in ocular angiogenesis, as well as in other angiogenesis diseases such as tumor growth, have validated the value of the VEGF pathway for clinical effect, the experimental agents are far from effective for many patients. It is clear that better inhibitors of the VEGF pathway are needed if we are to develop treatments for these major eye diseases.

### SUMMARY OF INVENTION

The present invention provides compositions and methods for use of RNAi agents, including small interfering RNA or siRNA (double stranded RNA oligonucleotides), and delivery systems to inhibit expression of pro-angiogenic factors and as a result inhibit ocular NV disease.

It is therefore an object of the invention to provide a composition comprising at least one dsRNA oligonucleotide and a pharmaceutical carrier, wherein upon administration to a subject suffering from an ocular disease associated with neovascularization or angiogenesis said dsRNA inhibits expression of a gene associated with neovascularization or angiogenesis in an ocular disease.

It is a further objection of the invention to provide a method for treating ocular disease in a subject, wherein said disease is characterized at least in part by neovascularization, comprising administering to said subject a composition comprising a dsRNA oligonucleotide and a pharmaceutically acceptable carrier, wherein said dsRNA oligonucleotide inhibits expression of a gene that promotes ocular neovascularization in said subject.

In one embodiment the pharmaceutical carrier is selected from the group of a polymer, lipid, or micelle. The carrier may be, for example, selected from the group consisting of polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer.

The ocular disease may be is selected from the group consisting of stromal keratitis, uveitis, rubeosis, conjunctivitis, keratitis, blepharitis, sty, chalazion, iritis, macular degeneration, and retinopathy. The ocular disease may be in at least the anterior of the eye. The composition may be administered at a site distal to the eye, for example, selected from the group of subconjunctival, intravenous, and subcutaneous administration and/or the composition may be administered topically to the eye.

In another embodiment, the dsRNA inhibits expression of a gene selected from the group of pro-inflammatory pathway genes, pro-angiogenesis pathway genes, pro-cell proliferation pathway genes, and viral infectious agent genome RNA, and viral infectious agent genes. The composition may comprise at least two dsRNA molecules, where each dsRNA molecule inhibits expression of a gene selected from the group of pro-inflammatory pathway genes, pro-angiogenesis pathway genes, pro-cell proliferation pathway genes, and viral infectious agent genome RNA, and viral infectious agent genes. The composition may comprise at least. three dsRNA molecules wherein at least one dsRNA molecule inhibits expression of VEGF, at least one dsRNA molecule inhibits expression of VEGF R1, and at least one dsRNA molecule inhibits expression of VEGF R2. The composition may comprise at least two dsRNA molecules wherein at least one dsRNA molecule inhibits expression of basic FGF and at least one dsRNA molecule inhibits expression of FGF R.

The dsRNA molecules may inhibit expression of one or more VEGF pathway genes, FGF pathway genes, or a combination thereof The dsRNA molecules may inhibit expression one or more pro-angiogenesis genes, pro-inflammatory genes, or a combination thereof. The dsRNA molecules may inhibit expression of one or more pro-angiogenesis genes, herpes simplex virus genes, or a combination thereof. The dsRNA molecules may inhibit expression of one or more pro-angiogenesis genes, endothelial cell proliferation genes, or a combination thereof The dsRNA molecules may inhibit expression of one or more pro-inflammation genes, herpes simplex virus genes, or a combination thereof.

The composition may comprise at least three dsRNA molecules that inhibit expression bind of at least two or more genes. The genes may encode VEGF, VEGF R1, and VEGF R2, basic FGF, FGF R, and/or combinations thereof. The genes may be pro-angiogenesis genes, endothelial cell proliferation genes, herpes simplex virus genes, pro-inflammatory genes, or a combination thereof.

In these compositions the dsRNA molecule may be a dsRNA oligonucleotide.

### BRIEF DESCRIPTION OF THE FIGURES

FIG. 1 shows a schematic structure of TargeTran™ (TT) containing three functional domains: a cationic core, a steric polymer, and a peptide ligand. TT electrically interacts with nucleic acids, and initiates self-assembly into nanoparticles that deliver the payloads selectively to cells with the receptor of the ligand.

FIG. 2 shows siRNA-mediated in vitro knockdown of VEGF-pathway genes RAW264.7 gamma NO (−) cells (A) and SVR cells (B) in 35 mm wells were transfected with siRNA targeting mVEGFA and mVEGFR1, respectively, at the amount indicated. 293 cells (C) were cotransfected with siRNA targeting mVEGFR2 and plasmid expressing mVEGFR2 at the amount indicated. Cellular RNA was isolated 48 h post-transfection, and the knockdown of endogenous expression of mVEGFA or mVEGFR1, or exogenous expression of mVEGFR2 was measured by RT-PCR for mVEGFA, or RS-PCR for mVEGFR1 and mVEGFR2.

FIG. 3 shows ocular delivery of FITC-labeled siRNA Six hours after CpG implantation mice were given FITC-Labeled siRNALuc through local (left) or systemic (right) routes, along with PT or TT, respectively. One dose of 10 μg siRNA per eye (local delivery) or 40 μg per tail (systemic delivery) was used. Cryosections of eyeballs, liver, and lung were examined under fluorescence microscope 24 hours after siRNA delivery. Only the result of the eye sections are shown.

FIG. 4 shows decreased level of VEGF mRNA in a cornea that was infected and treated with siVEGFmix with either local or systemic delivery Two corneas were collected on day 4 or 7 p.i. from mice that were infected with 1×10⁵ pfu HSV-1 RE and were treated with siRNAs targeting VEGF-pathway genes at day 1 and 3 p.i. by either local (10 μg/eye) or systemic (40 μg/tail) administration and then VEGF mRNA level was measured by RT-PCR (A) or quantitative real-time PCR (B).

FIG. 5 shows reduced levels of VEGF protein in a cornea that was infected and treated with siVEGFmix with either local or systemic delivery On day 7 p.i. 2 corneas/mouse were processed to measure the VEGF protein level. Levels of VEGF were estimated from supernatants of corneal lysates of mice infected with HSV-1 and treated with siRNAs targeting VEGF-pathway genes by an antibody capture ELISA as outlined in materials and methods. Results are expressed as mean±SD of four separate mice (2 corneas per mouse). Statistically significant differences in VEGF protein levels (p<0.05) were observed between the groups.

FIG. 6 shows local delivery of siRNAs targeting VEGF-pathway genes inhibits CpG ODN-induced angiogenesis. 24 h after implantation with CpG ODN (1 μg) into the micropocket in mouse cornea, mouse was given 10 μg per eye of siLacZ, siVEGFA, siVEGFR1, siVEGFR2, or siVEGFmix (an equal molar mixture of total siRNAs targeting VEGF-pathway genes) through subconjunctival injection along with PT. The angiogenesis area was measured on day 4 and 7 after the CpG pellets implantation (four mice per group). Statistically significant differences in angiogenic areas (* p<0.05, ** p<0.01) were observed between the groups (A). The NV was shown by the photos taken on day 7 (40×) (B).

FIG. 7 shows that systemic delivery of siRNAs against VEGF-pathway genes inhibits CpG DNA-induced angiogenesis. Individual siRNAs or a mixture of total siRNAs against VEGF-pathway genes were delivered with TT, 6 and 24 h after the CpG ODN induction by tail vein injection. The angiogenesis area was measured on day 4 and 7 after the CpG pellets implantation (four mice per group). Statistically significant differences in angiogenic areas (* p<0.05, ** p<0.01) were observed between the groups (A). The NV was shown by the photos taken on day 7 (40×) (B).

FIG. 8 shows that the TargeTran™-mediated systemic delivery enhanced the efficacy of siRNAs and exhibited dose-response 6 and 24 h after the CpG ODN induction a mixture of the tested antiangiogenic siRNAs, or the unrelated siLuc contral, were systemically delivered along with TT, respectively. siVEGFmix also was delivered with PBS. The angiogenesis area was measured on day 4 and day 7 post CpG-pellet implantation; and statistically significant differences (*p<0.05) were observed between these groups (four mice per group) (A). Dose-response experiment was performed, with mixed siRNAs targeting VEGF-pathway genes systemically delivered at 10, 20, 40, and 80 μg total siRNAs per mouse. The angiogenesis area was measured on day 4 and 7 post CpG-pellet implantation, and the anti-angiogenic efficiency was compared between different siRNA dosages (B).

FIG. 9 shows siRNA-mediated reduction of the severity of HSK and angiogenesis. Mice were infected with 1×10 pfu HSV-1 RE per eye, and on day 1 and day 3, p.i., were treated with siVEGF mix or siLuc, locally and systemically, respectively. The means of the HSK clinical scores (A) or clinical angiogenic scores (B) were calculated on day 10 p.i. Each dot represents the clinical score for one eye. Horizontal bars and figures in the parenthesis indicate the mean for each group. Data were collected from two separate experiments with 6 eyes from each group. Statistically significant differences in HSK or angiogenesis score (p<0.05) were observed between groups. On day 14 p.i. extensive growth of blood vessels and ulceration were seen in the infected cornea of siLuc-treated mice while the siVEGF mix-treated mice showed less NV close to the limbal area. Local and systemic deliveries showed obvious reduction of NV area A systemic delivery result is shown in a photo (C).

FIG. 10 shows tissue distribution of green fluorescently labeled siRNA in tumor bearing mice. Mice received 40 mg fluorescently labeled siRNA by intravenous injection as P-nanoplexes (left column), or RPP-nanoplexes (middle column) or aqueous solution (right column). One hour after injection, tissues were dissected and examined on a fluorescence microscope. Pictures were taken at equal exposure times for each tissue. P-nanoplexes show punctate fluorescence in all organs, especially high in lung and liver. RPP-nanoplexes show lower fluorescence levels in lung tissue with a punctate distribution and lower non-punctate fluorescence in liver. Higher levels of fluorescence were observed in the tumor as compared with P-nanoplexes. Fluorescence levels after administration of free siRNA were much lower in all organs, as compared with either of the two nanoplex formulations.

FIG. 11 shows inhibition of tumor neovascularization and VEGF R2 protein levels by intravenous administration of siRNA directed toward VEGF R2 complexed in nanoparticles. (B-D) Neovascularization in tumors treated with siRNA RPP-nanoplexes. Representative tumors excised at the end of the tumor growth inhibition experiment were examined using low magnification light microscopy. Trans-illumination of tumor and surrounding skin tissue shows strong neovascularization in mice left untreated (B) and mice treated with RPP-nanoplexes with siRNA-LacZ (C). In contrast, mice treated with RPP-nanoplexes with VEGFR2 siRNA showed low neovascularization and erratic branching of blood vessels (D). Asterisks indicate tumor tissue. Bar=2 mm. (E) VEGF R2 expression in tumor tissue after treatment with siRNA RPP-nanoplexes. Representative tumors removed at the end of the tumor growth inhibition experiment (A) were homogenized and VEGF R2-expression levels measured by western blotting. Left lane is untreated tumor, Middle lane is LacZ siRNA treatment and Right lane is VEGF R2 siRNA treatment.

FIG. 12 shows the green fluorescence measurement of leaky neovascularization in the retina in negative control siRNA treated eye (siLuc) versus inhibition of green fluorescence measurement from inhibition of leaky neovascularization in the retina of an eye treated with VEGF pathway active siRNA (siMix). The flatmounting reveals inhibition of NV by delivery of active siRNA inhibitors targeting VEGF, VEGF R1 and VEGF R2, but not the negative siRNA specific to Luciferase expression. The permeation of green fluorescent dye at sites of leaky neovascularization is observed by green in the image from the flat mount microscopic method. The image on the left from an eye treated With a negative control siRNA oligonucleotide, siLuc, shows. green fluorescence as a result of leaky neovascularization. The image on the right from an eye treated with the active siRNA oligonucleotide, siMix, shows a reduction in green fluorescence as a result of a reduction in leaky neovascularization, compared to the negative control.

### DETAILED DESCRIPTION

The present invention provides compositions and methods for treatment of eye neovascularization diseases such as Diabetic Retinopathy (DR), Age related macular degeneration (AMD), Uveitis, Stromal Keratitis (SK) and cancers. In one embodiment the invention uses RNAi-mediated inhibition of cells and biochemical pathways to achieve inhibition of eye diseases. The invention provides RNAi agents including siRNA oligonucleotides to inhibit 1) gene expression of viral infections, 2) inflammatory cells and biochemical pathways, 3) pro-angiogenesis cells and biochemical pathways including VEGF, VEGF receptors, FGF, FGF receptors, PDGF, and PDGF receptors, 4) cell proliferation mediating ocular neovascularization, and 5) their combination. In one embodiment, the invention uses systemically administered, chemically synthesized carriers that provide delivery of synthetic siRNA oligonucleotides. The invention provides for siRNA-mediated antiangiogenic effects localized at ocular tissues and at tissues with neovascularization disease. The invention provides methods for nucleic acid agents, proteins or peptides, and for small molecules to inhibit excessive neovascularization eye diseases. The invention also provides for combinations of agents that provide inhibition of the multiple factors and the multiple biochemical pathways that induce unwanted ocular neovascularization. The invention also provides clinical means for delivery of therapeutic agents to ocular tissues. The methods and compositions of the invention are useful for treatment of ocular neovascularization resulting from eye infections, diabetic retinopathy, age-related macular degeneration, and eye cancer.

VEGF is an essential growth factor responsible for normal vasculagenesis and angiogenic remodeling. Under some disease conditions VEGF angiogenic pathway will be activated, such as in the situation of tumors where new blood vessels are formed to deliver enough oxygen and nutrition to the rapidly growing abnormal tissues. The majority of severe visual loss in the United States results from complications associated with retinal neovascularization in patients with ischemic ocular disease such as diabetic retinopathy, retinal vein occlusion, and retinopathy of prematurity. Intraocular expression of the angiogenic protein VEGF is closely correlated with neovascularization in these human disorders and with ischemia-induced retinal neovascularization in mice. Therefore, the VEGF pathway composed of VEGFs and VEGF receptors, is a logical target for inhibition of retinal angiogenesis.

To evaluate anti-angiogenic agents for development of novel therapeutics for ocular NV diseases, published clinically relevant animal models are available. One clinically relevant model for retinal angiogenesis uses hypoxia to induce excessive angiogenesis. Another model for retinal angiogenesis uses laser bums to create lesions in the retina into which angiogenesis occurs. One clinically relevant model for corneal NV uses disease induction either by CpG previously implanted in the micropocket in mice cornea stroma or HSV infection, and through which the inhibition of angiogenesis can easily be measured. The effects of candidate therapeutics can be first tested in cell culture and then selected for studies in clinically relevant animal models of disease.

### RNAi Therapeutic Approach

RNAi, the double stranded RNA (dsRNA)-induced sequence-specific degradation of messenger RNA (mRNA), often called gene silencing, has been proven to be a powerful tool for gene discovery or gene validation, and it holds great potential in developing novel gene-specific drugs. In our anti-angiogenic RNAi design for the inhibition of eye NV, mVEGF-A, mVEGF-R1, and mVEGF-R2 are chosen to be the target genes that are key players in the VEGF angiogenic pathway. Small interfering RNAs (siRNAs) have been designed according to general guidelines proposed by Tuschl's research team. The siRNAs are 21-nucleotide long double stranded RNAs with 2-nt overhangs at either 3′ termini, with the negative strand complementary to the targeted mRNA sequences. The knockdown of these genes, singly or in combination, has the impact of blocking the angiogenic pathway leading to the inhibition of NV, and thus the relief of the SK symptoms. The same methodology applies to other NV-related ocular diseases.

To date, no suitable technology has been reported for RNAi agent delivery to target ocular neovascularization. Therefore, a strong need exists for proprietary nucleic acid delivery systems for RNAi agents for ocular neovascularization diseases. Use of RNA interference (RNAi) has been developing rapidly in cell culture and model organisms such as Drosophila, C. elegans, and zebrafish. Studies of RNAi have found that long dsRNA is processed by Dicer, a cellular ribonuclease III, to generate duplexes of about. 21 nt with 3′-overhangs, called short interfering RNA (siRNA), which mediates sequence-specific mRNA degradation (4, 5, 6). Understanding the mechanisms of RNAi and its rapidly expanding application represents a major breakthrough during the last decade in the field of biomedicine. Use of siRNA duplexes to interfere with expression of a specific gene requires knowledge of target accessibility, but is blocked by a lack of effective delivery of the siRNA into the target cells for ocular NV diseases. Along with the fast growing literature on siRNA as a functional genomic tool, there is emerging interest in using siRNA as a novel therapeutic. Therapeutic applications clearly depend upon effective local and systemic delivery methods. The advantages of using siRNA as a therapeutic agent are clearly due to its specificity, stability, potency, natural mechanism of action, and uniform chemical nature of agents targeting different gene targets since they differ only in nucleotide sequence.

We have used siRNA to silence the pro-angiogenic factors in tumor models and have demonstrated strong gene silencing effects (10). This progress demonstrated the feasibility and efficacy of RNAi silencing of pro-angiogenic factors in vitro and in vivo, and proved that our molecular design and proprietary delivery system are applicable for siRNA-mediated gene silencing to treat ocular neovascularization. A systemic delivery system, known as TargeTran™ has been used for siRNA delivery (11). The siRNA delivery carrier is a cationic polymer based technology described in Woodle et al. (WO 01/49324, the contents of which are hereby incorporated by reference in their entirety). As used herein, a “synthetic vector” means a multi-functional synthetic vector which, at a minimum, contains a nucleic acid binding domain and a ligand binding (e.g. tissue targeting) domain, and is complexed with a nucleic acid sequence. A synthetic vector also may contain other domains such as, for example, a hydrophilic polymer domain, endosome disruption or dissociation domain, nuclear targeting domain, and nucleic acid condensing domain. A synthetic vector for use in the present invention preferably provides reduced non-specific interactions, yet effectively can engage in ligand-mediated (i.e. specific) cellular binding. In addition, a synthetic vector for use in the present invention is able to be complexed to one or more therapeutic nucleic acids, which then can be administered to a subject. We have administrated these siRNAs in rodents with this systemic approach by tail vein injection. The knockdown effects for VEGF A, VEGF R1 and VEGF R2 resulted in significant inhibition of ocular neovascularization (13).

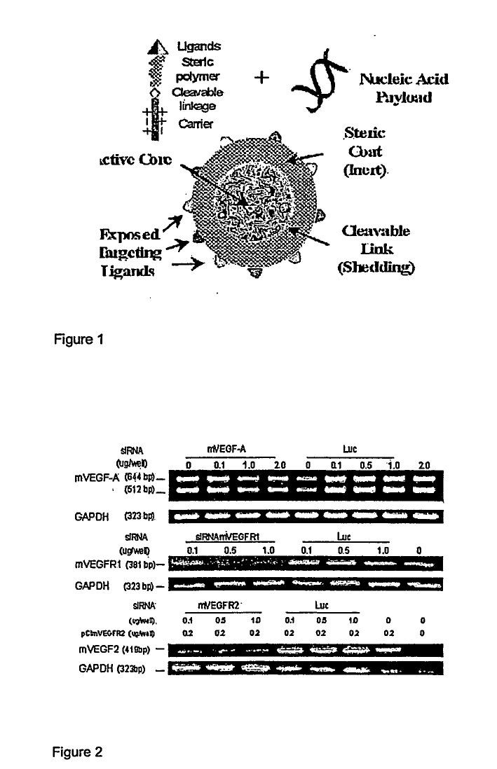

We also have used another polymer-based carrier known as PolyTran™, to deliver siRNAs in vitro and in vivo. This technology (see WO 0147496, the contents of which are hereby incorporated by reference in their entirety) is able to substantially reduce the formation of neovasculature induced by CpG in the otherwise avascular mice eye cornea. Our success in the siRNA design and experimentation exhibit the great possibility of developing RNAi therapeutics to cure herpetic SK and other angiogenic eye diseases (FIG. 1-3).

### RNAi and Therapeutic Agents

The invention provides interfering RNA agents that inhibit gene expression and intervene in ocular neovascularization. The invention provides many forms of interfering RNA molecules as therapeutic agents, including double stranded RNA (dsRNA) oligonucleotides, small-hairpin RNA (shRNA), and DNA-derived RNA (ddRNA). The invention also provides nucleic acid agents active according to their sequence but not necessarily considered “RNA interference”, including decoy oligonucleotides, antisense, ribozymes, gene expression, and aptamers. These nucleic acids and other therapeutic agents carry a net negative charge, or other physical property, such that the formulation and compositions of the invention provide contact, and uptake when required, by ocular tissues and cells.

RNAi is a potent method that can be used to knock down gene expression, destroying an mRNA in a sequence-specific manner. RNAi can be managed to provide biological function in a rapid and sustained fashion. The present invention provides RNAi agents giving a gene selective intervention to treat ocular NV or other NV-related eye diseases, as a means to control human eye diseases.

Design of Interfering RNAs

The RNAi agents are designed to have a nucleotide sequence matching a portion of the sequence of a targeted gene. The selected RNAi sequence of the targeted gene may be in any part of the mRNA generated by expression of the gene. The RNAi comprises a sequence that will hybridize with mRNA from the target gene, an “antisense strand” of the RNAi sequence. The RNAi sequence comprises a sequence that will hybridize with the antisense strand, a “sense strand” of the RNAi sequence. The RNAi sequence selected of the targeted gene should not be homologous with any other mRNA generated by the cell, nor with any sequence of the targeted gene that is not transcribed into mRNA. Numerous design rules for selecting a sequence of 20 to 27 bases of the target mRNA sequence are known, including commercially available methods. These design methods evolve and the most current available methods can be used. Designs can be obtained from at least three methods and a single consensus list of highest priority constructed and assembled from these methods. The inventors have found that preparation of at least 6 of the highest priority candidate sequences, followed by cell culture testing for gene inhibition nearly always reveals at least two active RNAi sequences. If not, a second round of obtaining six highest priority candidate sequences and testing can be used.

Besides identification of active RNAi sequences, the design also must ensure homology only with wanted mRNA sequences. A poor homology of RNAi sequences with genomic sequences other than those of the target gene mRNA reduces off-target effects at either the mRNA level or the gene level. Also, a poor homology of the “sense strand” of the RNAi sequence reduces off-target effects. By DNA comparison with Clone Manager Suite (SciEd Software, Cary N.C.) and by an on-line Blast search, the targeted sequences of the selected gene can be confirmed to be unique and to lack sequence homology for other genes including human counterparts. For example, sequences matching the mRNA of mVEGF-A are confirmed to be unique for mVEGF-A without homology for mVEGF-B mRNA, mVEGF-C mRNA, mVEGF-D mRNA, or human counterparts including hVEGF165-a (AF486837). However, the matching sequences will target multiple isoforms of mVEGF-A, e.g., mVEGF (M95200), mVEGF115 (U502791), mVEGF-2 (S38100), mVEGF-A (NM_{—}192823), that encode mVEGF-A proteins of 190 amino acid (aa), 141 aa, 146 aa, and 148 aa, respectively. All of the published cDNA sequences of these mVEGF-A isofoms, except mVEGF-A (NM_{—}192823, a mature form of protein), include a 26-aa signal peptide at the N-terminus. The targeted sequences of mVEGF are chosen not in the signal peptide part, but in the mature protein part shared by all these mVEGF-A isoforms. Targeted sequences of mVEGF-R1 and mVEGF-R2 are also confirmed to be unique for these two genes, respectively. Different forms of interfering RNAs are included in present invention. As an example, the small interfering RNAs, siRNAs, are designed according to the above target sequences, using known guidelines. These siRNAs are 21-nt double stranded RNA oligos with 2 nts (TT) at 3′ overhangs. The targeted sequences (mRNA sequences) and the sequences of siRNAs are listed in Table 1.

The RNAi agents are specific for the target gene sequence, which is dependent upon the species of the gene. Most mammalian genes share considerable homology where RNAi agents can be selected to give activity for genes in all species with that homologous segment of the gene mRNA. The preferred RNAi agent design of the invention has perfect homology with the human gene mRNA sequence and a sufficient homology with the gene mRNA of at least one animal species used for toxicity testing to give activity toward that animal species gene.

The RNAi agents are specific for a target gene sequence, which can include specificity for a single nucleotide polymorphism, SNP. The preferred RNAi agent design of the invention has perfect homology with all human gene mRNA sequences of the target gene of all polymorphisms related to disease pathology and has reduced homology with all human gene mRNA sequences of the target gene of all polymorphisms unrelated to disease pathology.

TABLE 1
Genes and some targeted mRNA sequences (antisense strands shown)

| | | |
|---|---|---|
| Genes | Targeted sequences (5′-3′) | Notes |

| | | | |
|---|---|---|---|
| mVEGF-A | 1 | AAGCCGTCCTGTGTGCCGCTG | 91-111 nt of cds, or 151-171 |
| | | | or XM_192823 sequence. |
| | 2 | AACGATGAAGCCCTGGAGTGC | 133-153 nt of cds, or 193-213 |
| | | | nt of XM_192823 sequence. |
| mVEGFR-1 | 1 | AAGTTAAAAGTGCCTGAACTG | 82-102 nt of cds, or 333-353 |
| | | | nt of D88689 |
| | 2 | AAGCAGGCCAGACTCTCTTTC | 131-151 nt of cds, or 382-403 |
| | | | nt of D88689 |
| mVEGFR-2 | 1 | AAGCTCAGCACACAGAAAGAC | 97-117 nt of cds, or 304-324 |
| | | | nt of NM_010612 |
| | 2 | AATGCGGCGGTGGTGACAGTA | 233-243 nt of cds, or 440-460 |
| | | | nt of NM_010612 |

Clinically Relevant Animal Models

A recent mouse eye model presents a feasible and clinically relevant model for corneal NV. In this model, purified HSV DNA (CpG rich) and/or synthetic CpG motif-oligonucleotide (CpG ODN) are used to induce VEGF expression in cornea, thus inducing NV, and corneal SK, instead of using HSV infection or VEGF proteins. In this model, the new blood vessel formation is readily induced and measured. The present invention employs this model to test the interfering RNAs and to collect data on the RNAi therapy of the CpG-induced SK, providing quantitative data, and producing an environment close to that of HSV infection-related eye SK in human.

Inhibition of Viral Infection

One of the well established causes of ocular neovascularization is herpes and other viral infections. A means to intervene in ocular neovascularization derived from viral infections is to inhibit the originating viral infection. The RNAi agents utilize an endogenous process active against dsRNA viral infections but can be used to inhibit expression from virtually any mRNA, and with a high degree of selectivity. The invention provides for RNAi agents for inhibiting ocular viral infections as a means to intervene in ocular neovascularization. The RNAi agents of the invention include short dsRNA oligonucleotides, siRNA, with a sequence matching viral gene sequences and lacking sequence specificity for human genes. The RNAi agents of the invention inhibit mRNA expressed by either DNA or RNA viral infections and they degrade the genome of dsRNA viral infections. One DNA viral infection inhibited by the RNAi agents of the invention is HSV, which causes herpetic stromal keratitis. This virus has a relative large genome that remains episomal and where expression levels of viral mRNA rise and fall over time. The continuous low levels of HSV viral mRNA expression result in a persistent, albeit quiescent, infection that flairs up from time to time. The RNAi agents of the invention are useful to inhibit rising HSV mRNA expression associated with recurrence of infection. By reducing the ability of the infection to flair up, the RNAi agents protect from induction of ocular neovascularization disease. The RNAi agents also are useful to diminish the continuous, low level HSV mRNA expression to even lower levels, which diminishes the ability of the HSV infection to flair up. The RNAi agents are effective to inhibit the DNA and RNA viral infections of ocular tissues that lead to ocular neovascularization.

Inhibition of Inflammatory Cells and Pathways Stimulating Ocular NV

Inflammation is a process that involves many cells and biochemical factors, but despite its complexity the process is highly conserved across tissues. One of the early events in inflammation is secretion of activating factors as a result of tissue hypoxia, damage, or other insults. These factors activate cells and induce recruitment of inflammatory cells into the tissue, which secrete additional activating factors. One common biochemical pathway for induction of inflammation is secretion of TNF and IL-1. These factors act in a largely parallel manner so that strong inhibition of their activation of an inflammation cascade requires intervening in both simultaneously. Downstream of this point, the inflammatory cascade results in secretion of factors to induce neovascularization. The inflammatory process offers many points for intervention: upstream at secreted factors initiating the cascade; and downstream at factors responsible for activating specific cells in the cascade, such as endothelial cell recruitment of neutrophils from the blood and endothelial cell induction of neovascularization. The invention provides RNAi agents effective for inhibiting factors whose upregulation and role in inflammation depends on gene expression of the factor. While many secreted factors are present in cells and released to initiate inflammation, up regulation of expression of those same factors is important for continued expansion of the inflammation and for persistent of the inflammation. The RNAi agents of the invention provide for inhibition of persistent inflammation, which is a greater contributor to the ocular neovascularization disease. Numerous factors are involved in the inflammation pathway and specifically for the persistent ocular inflammation that leads to ocular neovascularization disease, including and importantly endothelial cell activation.

Inhibition of Angiogenic Pathways Mediating Ocular NV

The angiogenesis process, like inflammation, is complex but highly conserved across tissues. Another similarity is the major role several secreted factors play. An early step is driven by the VEGF pathway that involves secretion of VEGF growth factors, which bind and activate cells bearing different members of the VEGF family of receptors. These growth factors also interact with other receptors, e.g. NP-1, that stimulate the cells bearing them, including tumor cells. Another major secreted angiogenic growth factor is bFGF, which actives a separate set of receptors. Both of these pathways activate endothelial cells in nearby vasculature, and stimulate their proliferation and migration to form new vasculature into the region secreting the growth factor stimulants. However, the intracellular kinase signal transduction pathways induced by the VEGF and bFGF pathways merge at a common point related to c-RAF or its downstream target transcription factors such as NFkB. Thus the VEGF and bFGF pathways act somewhat in parallel up to a point where they become the same. These secreted growth factor pathways of neovascularization represent a very useful point for therapeutic intervention, as provided by the invention, either by inhibiting the growth factors or their receptors, or both. The invention also provides for inhibiting both pathways simultaneously, as well as for inhibition of intracellular signaling induced by these pathways such as the induced signal transduction kinases, or in a preferred embodiment the transcription factors. The transcription factors have been established as useful points for therapeutic intervention but have been intractable to conventional therapeutic modalities. The invention provides for RNAi agents inhibiting expression of proteins, including transcription factors that now enables therapeutic intervention at these key intracellular steps of neovascularization.

The VEGF family is composed of five structurally related members: VEGF-A, Placenta Growth factor (PIGF), VEGF-B, VEGF-C, and VEGF-D. There are three structurally homologous tyrosine kinase receptors in the VEGF receptor family: VEGFR-1 (Flt-1), VEGFR-2 (KDR or Flk-1), and VEGFR-3 (Flt4), with different affinity or functions related to different VEGF members. While function and regulation of other four VEGF members are less understood, VEGF-A, which binds VEGFR-1 and VEGFR-2, is known to induce neovascularization, angiogenesis, and vascular permeability. In order to functionally interact with their specific receptors VEGF naturally forms homo-dimers. VEGFR-1 and VEGFR-2 are both up-regulated in tumor and proliferating endothelium that may be a direct response to VEGF-A or partly to hypoxia. It is well accepted that VEGFR-2 mediates angiogenic signals for blood vessel growth, and is necessary for proliferation. However, the function of VEGFR-1 is less well characterized. VEGF-R1 has higher affinity to VEGF-A than VEGFR-2, and mediates motility and permeability, therefore plays a role in transducing angiogenic signals. The understanding of basic biology of the VEGF and VEGF receptors provides solid foundation for the design of approaches to target the VEGF signaling pathway. The invention provides RNAi agents specific for inhibition of murine and human forms of VEGF, the VEGF receptors, and intracellular signal transduction pathway.

The bFGF pathway is one of several FGF pathways. The bFGF factor is a strong stimulator of angiogenesis and thus it and its receptors are an important point for therapeutic intervention. The invention provides RNAi agents specific for inhibition of bFGF and its receptors, and intracellular signal transduction pathway.

Inhibition of Ocular NV Endothelial Cell Proliferation

A key step in formation of neovasculature is proliferation of activated endothelial cells in nearby vasculature. This step is an important point for therapeutic intervention. Many intracellular factors are well known to be critical for endothelial cell survival and/or proliferation. The two embodiments provided by the invention are 1) block endothelial cell proliferation and 2) induce apoptosis in activated endothelial cells. Either or both of these embodiments result in a reduction in neovasculature due to inhibition of endothelial cell proliferation and migration. The invention provides for RNAi agents inhibiting cell cycle, which inhibits proliferation. The invention also provides for RNAi agents initiating activated endothelial cell apoptosis. The invention provides ligand targeted nanoparticles that selectively bind activated endothelial cells through αvβ3/αvβ5 integrin up-regulation associated with neovasculature activation. The nanoparticles provided by the invention deliver the RNAi into the intracellular compartment of the endothelial cells, which induce apoptosis or inhibit proliferation, or both.

Combined Modalities to Inhibit Ocular NV Diseases

The processes leading to excessive and unwanted ocular neovascularization are complex and generally involve parallel biochemical pathways. As a result, therapeutic intervention at one target or even one pathway can be incomplete in control of disease pathology (neovascularization). The invention provides for combined intervention: intervention in multiple targets of a biochemical pathway or intervention in multiple pathways or both. For example, the invention provides for intervention with multiple targets of the VEGF pathway including a combination of siRNA for VEGF-A, VEGF-R1, and VEGF-R2. The invention further provides for intervention in multiple pathways including a combination of siRNA VEGF pathway targets and bFGF pathway targets. The invention also provides for combinations of these combinations, e.g. combined siRNA for VEGF pathway and bFGF pathway. The invention also provides for combined intervention in multiple aspects of the disease pathology, such as initiating factors including viral infectious agents, initiating inflammation pathways, angiogenic pathways, and endothelial cell proliferation pathways.

Delivery of Therapeutic Agents Including Local, Topical, and Systemic

The invention provides compositions and methods for administering the therapeutic agents to treat ocular neovascularization diseases, and in particular to treat diseases in the anterior of the eye. The invention also provides compositions and methods for administering the therapeutic agents to treat ocular neovascularization diseases anywhere in the eye including the posterior of the eye. The tissues anywhere in the eye can be treated with neovasculature-targeted delivery of therapeutic agents, according to the invention, by local administration, by topical administration to the eye, and by intravenous administration at a distal site. The tissues in the anterior of the eye can be treated, according to the invention, by local administration into the subconjunctival tissue, by topical administration to the eye, by periocular injection, by intraocular injection, and by intravenous administration at a distal site. The compositions provided by the invention include 1) cationic agents that bind nucleic acids by an electrostatic interaction, including non-natural synthetic polymers, grafted polymers, block copolymers, peptides, lipids and micelles, 2) hydrophilic agents that reduce non-specific binding to tissues and cells, including non-natural synthetic polymers, peptides, and carbohydrates, 3) tissue and cell penetrating agents, including surfactants, peptides, non-natural synthetic polymers, and carbohydrates.

A preferred class of peptide is the histidine-lysine copolymer that is a basic, cationic, broad class of peptides, referred to in some instances as PolyTran™. Another preferred class of peptide is linear polylysine with histidine or imidazole monomers coupled to the epsilon amino moiety of the lysine. monomers. Another preferred class of peptide is branched polylysine and branched polylysine with histidine or imidazole monomers coupled to the epsilon amino moiety of the lysine monomers. A preferred composition has a self-assembled complex of negatively charged therapeutic agent such as a nucleic acid with a cationic peptide with an excess of cationic charge of 2 fold to 10 fold and a more preferred cationic charge of 2 fold to 6 fold. A preferred class of polylysine coupled with histidine or imidazole monomers has 30 to 70% coupling to primary amines of the lysine monomers. Another preferred class of peptide is a polymer with a monomer comprised of the tripeptide histidine-histidine-lysine or the tetrapeptide of histidine-histidine-lysine-lysine, where the polymer is either linear or branched, the branched polymer having monomers coupled to either the alpha or epsilon amino group of another monomer, or both. A preferred molecular weight of the polylysine class of polymers is in the range of 5,000 to 100,000, and a more preferred molecular weight of 10,000 to 30,000.

A preferred class of grafted polymers is a peptide grafted with a hydrophilic polymer, where the hydrophilic polymer includes PEG, polyoxazoline, polyacetal (referred to in some instances as Fleximer), HPMA, and polyglycerol. A preferred composition has a self-assembled complex of negatively charged therapeutic agent such as a nucleic acid with a cationic grafted polymer with an excess of cationic charge of 2 fold to 10 fold and a more preferred cationic charge of 2 fold to 6 fold. A preferred molecular weight of the hydrophilic polymer is in the range of 2,000 to 10,000. Another preferred class of grafted polymers is a peptide grafted with a hydrophilic polymer further comprised of a ligand grafted to the hydrophilic polymer, where the ligand includes peptides, carbohydrates, vitamins, nutrients, and antibodies or their fragments.

A preferred class of non-natural synthetic cationic polymer is a polymer with a backbone repeating unit of ethyl-nitrogen (—C—C—N—), including polyoxazoline and polyethyleneimine (PEI). A preferred composition has a self-assembled complex of negatively charged therapeutic agent such as a nucleic acid with a cationic polymer with an excess of cationic charge of 2 fold to 10 fold and a more preferred cationic charge of 2 fold to 6 fold. In one embodiment, the invention provides linear polyoxazoline or PEI derivatized with histidine or imidazole monomers. Another preferred class of polymer is branched polyoxazoline or PEI derivatized with histidine or imidazole monomers. A preferred class of polymer coupled with histidine or imidazole monomers has 30 to 70% of the basic moieties being imidazole. A preferred molecular weight of the polymers is in the range of 5,000 to 100,000, and a more preferred molecular weight of 10,000 to 30,000.

A preferred class of grafted polymers is a polymer grafted with a hydrophilic polymer, where the hydrophilic polymer includes PEG, polyoxazoline, polyacetal (referred to in some instances as Fleximer), HPMA, and polyglycerol. A preferred composition has a self-assembled complex of negatively charged therapeutic agent such as a nucleic acid with a cationic grafted polymer with an excess of cationic charge of 2 fold to 10 fold and a more preferred cationic charge of 2 fold to 6 fold. Another preferred class of grafted polymers is a polymer grafted with a hydrophilic polymer further comprised of a ligand grafted to the hydrophilic polymer, where the ligand includes peptides, carbohydrates, vitamins, nutrients, and antibodies or their fragments.

Another preferred class of cationic polymer is a polymer with a polyacetal backbone. A preferred composition has a self-assembled complex of negatively charged therapeutic agent such as a nucleic acid with a cationic polyacetal polymer with an excess of cationic charge of 2 fold to 10 fold and a more preferred cationic charge of 2 fold to 6 fold. In one embodiment, the invention provides linear polyacetal derivatized with a basic moiety, where the basic moiety class includes mixture of lysine, primary amine, histidine, and imidazole monomers. Another preferred class of polymer is branched polyacetal derivatized with a basic moiety (again including the class of lysine, amine, histidine, and imidazole monomers). A preferred class of polyacetal polymer coupled with lysine, amine, histidine, and imidazole monomers has 30 to 70% if the basic moieties being imidazole. A preferred molecular weight of the polymers is in the range of 5,000 to 100,000, and a more preferred molecular weight of 10,000 to 30,000. A preferred class of grafted polymers is a polymer grafted with a hydrophilic polymer, where the hydrophilic polymer includes PEG, polyoxazoline, polyacetal (referred to in some instances as Fleximer™), HPMA, and polyglycerol. Another preferred class of grafted polymers is a polyacetal polymer grafted with a hydrophilic polymer further comprised of a ligand grafted to the hydrophilic polymer, where the ligand includes peptides, carbohydrates, vitamins, nutrients, and antibodies or their fragments.

A preferred class of lipid is a substituted ethanol amine, as disclosed in Woodle et al. (WO 01/49324, the contents of which are hereby incorporated by reference in their entirety). A preferred composition has a self-assembled complex of negatively charged therapeutic agent such as a nucleic acid with a cationic lipid with an excess of cationic charge of 2 fold to 10 fold and a more preferred cationic charge of 2 fold to 6 fold. Another preferred class of lipid is a polymer grafted lipid with a hydrophilic polymer and yet another preferred class of lipid is a polymer grafted lipid further comprised of a ligand grafted to the hydrophilic polymer, where the ligand includes peptides, carbohydrates, vitamins, nutrients, and antibodies or their fragments.

A preferred class of micelle is a block copolymer with one block comprised of a hydrophilic polymer and another block comprised of a hydrophobic polymer, including polypropylene oxide, a hydrophobic polyoxazoline, a hydrophobic polymer derivatized with primary amines or imidazole or both, a hydrophobic polymer derivatized with a moiety that forms a cleavable linkage with the therapeutic agent including a sulfydryl for a disulfide, an aldehyde for a Schiff's base, and an acid or alcohol for an ester. A preferred composition has a self-assembled complex of negatively charged therapeutic agent such as a nucleic acid with a micelle with an excess mass of micelle over that of the therapeutic agent of 2 fold to 50 fold and a more preferred excess mass of 4 fold to 20 fold. Another preferred class of micelle is a block copolymer further comprised of a ligand grafted to the hydrophilic polymer, where the ligand includes peptides, carbohydrates, vitamins, nutrients, and antibodies or their fragments.

One embodiment of the invention comprises a polymer conjugate that consists of three functional domains: a cationic polymer such as 25kD PEI, a hydrophilic polymer such as 3.4 kD polyethylene glycol or PEG, and a ligand such as a disulfide stabilized, folded RGD-peptide. The cationic polymer domain condenses the nucleic acid (DNA or RNA) as employed in routine transfection of cultured mammalian cells. The hydrophilic polymer domain protects nucleic acids being delivered from degradation, and also shields the surface charge thus preventing non-specific charge-mediated interactions between the nucleic acid and proteins on cellular surface or existing in blood stream. These non-specific interactions often induce adverse distribution of the therapeutic agent or adverse biological activity. The RGD peptide ligand, the third domain, provides tissue and cell-specific targeting to cell surface integrins up-regulated in tissues where new blood vessels are formed, such as αvβ3 and αvβ5 integrins. This embodiment can be used for systemic siRNA delivery to target neovasculature and inhibit unwanted angiogenesis, including in eyes.

The invention provides for formulated delivery of therapeutic agents including siRNA into cells and tissues. The formula provides protection of nucleic acids from degradation and facilitates the tissue and cellular uptake of the therapeutic agent. By delivering RNAi or other negatively charged therapeutic agents with a formulation composition of the invention, the invention achieves cellular uptake of the therapeutic agent and inhibition of expression of an endogenous target gene. By the use of a formulation for local administration, the invention provides for local administration of siRNA and other therapeutic agents into eyes for treatment of ocular disease, including intrastromal infections, corneal neovascularization, stromal keratitis, uveitis, etc. Although local administration may be more invasive than distant systemic delivery, and incurs risk of infection or irritation leading to inflammation, the local delivery of siRNA may be preferred in many clinical situations, e.g., in severe NV conditions or in fast growing tumors. The invention also provides for incorporation of agents that increase the tissue adhesion and permeability through the corneal epithelium. This combination provides topical application in the form of eye-drops.

By use of the compositions and methods of the invention, siRNA and other therapeutic agents are used to treat ocular neovascularization by topical, local injection, or I.V. injection administration.

### EXAMPLES

### Example 1

### Local and Systemic Administration of VEGF Pathway Inhibitors to Treat Anterior Ocular NV Disease

Mouse SK Model, Virus, and Tissue Culture.

The ocular stromal keratitis (SK) BALB/c mouse models were previously reported where cornea NV was induced either by CpG DNA oligo (CpG ODN), that contained the equivalent NV-inducing motif in HSV DNA genome, implanted into stroma through micropocketing procedure, or by HSV-1 viral infection through corneal scarification. The sequences of stimulatory ODNs used in this study were: 1466, TCAACGTTGA, and 1555, GCTAGA CGTTAGCGT (provided by Dr. Dennis M. Klinman, Biologics Evaluation and Research, FDA, U.S.A.). The pellet to be used for implantation into the corneal micropocket contained an equal molar mixture of ODNs 1466 and 1555, along with hydron polymer as reported previously. HSV-1 strain RE (by Dr. Robert Lausch, Uni. Alabama, Mobile) was used in the induction of HSK at the dosage of 1×10⁵ plaque-forming units per eye in a 2-μl value. To test the in vitro RNAi effect, the following three cell lines were used: RAW264.7 gamma NO (−), ATCC, CRL-2278, a mouse macrophage cell line, expressing endogenous mVEGF-A. SVR, ATCC CRL-2280, a mouse endothelial cell. line bearing receptors for mVEGF (mVEGFR1 and mVEGFR2). 293 cell line, to be transfected with plasmid pCImVEGFR2 expressing mVEGFR2 driven by cmv promoter, for the detection of the knockdown of exogenous mVEGFR2.

Small Interfering RNAs (siRNA).

Double-stranded siRNAs were designed to target the VEGF-pathway factors: mVEGF-A (XM_{—}192823), mVEGFR1 (D88689), and mVEGFR-2 (MN_{—}010612). Two target sequences were picked up from each gene. These sequences are (from 5′ to 3′): mVEGF-A 1): AAG CCGTCCTGTGTGCCGCTG; mVEGF-A 2): AACGATGAAGCCCTGGAGTGC; mVEGFR1 1): AAGTTAA AAGTGCCTGAACTG; mVEGFR1 2): AAGCAGGCCAGACTCTCTTTC; mVEGFR2 1): AAGCTCAGCAC ACAGAAAGAC; 2): AATGCGGCGGT GGTGACAGTA. For synthesis of unrelated siRNA controls, two target sequences each for LacZ (E00696) and firefly luciferase (Luc, AF434924) were also selected. They were: LacZ 1): AACAGTTGCGCAGCCTGAATG; LacZ 2): AACTTAATCGCCTTGCAGCAC; Luc 1): AA GCTATGAAACGATATGGGC; 2): AACCGCTGGAGAGCAACTGCA. Blast sequence searching confirmed the specificity of these siRNAs with their targeted sequences, and the mVEGF-A targets were designed to be shared by different mVEGF-A isomers. All siRNAs were custom-designed as 21-nt double stranded RNA oligos with 19-nt duplex in the middle and dTdT overhang at the 3′-end of either RNA strand, following the well-accepted guidelines proposed by Tuschl's group; and were synthesized by Qiagen. To get better RNAi effect, we routinely used a mixture of two double-stranded 21-nucleotide RNA duplexes targeting two different sequences on a single mRNA molecule.

RNA Template-Specific PCR (RS-PCR & RT-PCR).

The RS-PCR was performed for detection of mRNA knockdown by siRNAs in vitro. Cytoplasmic RNA was isolated by RNAwiz (Ambion, #9736) according to the manufacturer's instruction with additional DNAse treatment, and subjected to RS-PCR with specially designed primers. The mRNA-specific reverse primers for the RT reaction were all 47-mer oligos with the 5′-end 30-mer of unique sequence (called “TS1” sequence, indicated in uppercase below) linked to a 17-mer sequence unique for each mRNA molecule (in lower case below). They were (from 5′ to 3′):

1) mVEGFA Dn:
GAACATCGATGACAAGCTTAGGTATCGATAcaagctgcctcgccttg;
2): mVEGFR1 Dn:
GAACATCGATGACAAGCTTAGGTATCGATAtagattgaagattccgc;
3) mVEGFR2 Dn:
GAACATCGATGACAAGCTTAGGTATCGATAggtcactgacagaggcg.

The PCR assays for all the tested genes, that follow the RT assay, used the same reverse primer, TS1: GAACATC GATGACAAGCTTAGGTATCGATA. The forward primers for PCR, were 30-mer oligos, unique for each gene:

1) mVEGFA Up:
GATGTCTACCAGCGAA GCTACTGCCGTCCG;
2) mVEGFR1 Up:
GTCAGCTGC TGGGACACCGCGGTCTTGCCT;
3) mVEGFR2 Up:
GGCGCTGCTAGCTGTCGCTCTGTGGT TCTG.

The RT-PCR of the housekeeping gene GAPDH was used as a control for the RNA amount used in RS-PCR. An oligo dT primer (19-mer) was used for RT assay of GAPDH. The primers used for the PCR were 20-mer oligos:

1) GAPDH Up:
CCTGGTCACCA GGGCTGCTT;
2) GAPDH Dn:
CCAGCCTTCTCCATGGTGGT.

RT-PCR was also used according to protocol described previously. For the detection of mVEGF-A expression the primers used were 5′-GCGGGCTGCCTCGC AGTC-3′ (sense) and 5′-TCACCGCCTTGGCTTGTCAC-3′ (antisense).

Quantitative Real-time PCR.

QRT-PCR was performed using a DNA Engine Opticon (MJ Research Inc.). PCR was performed using SYBR Green I reagent (Qiagen, CA), according to the manufacturer's protocol. During the optimization procedures of the primers, 1% agarose gel analysis verified the amplification of one product of the predicted size with no primer-dimer bands. The absence of primer-dimer formation for each oligonucleotide set was also validated by establishing the melting curve profile. The semi-quantitative comparison between samples was calculated as follows: the data were normalized by subtracting the difference of the threshold cycles (C_{T}) between the gene of interest's C_{T} and the “housekeeping” gene GAPDH's C_{T} (gene of interest C_{T}−GAPDH C_{T}=ΔC_{T}) for each sample. The ΔC_{T} was then compared to the expression levels of the vector control sample (sample ΔC_{T}−vector ΔC_{T}). To determine the relative enhanced expression of the gene of interest, the following calculation was made: fold change=2^{(-sample ΔCT-vector ΔCT)}. The primers used were mGAPDH sense, 5′-CATCCTGCACCACCAACTGCTTAG-3′ and GAPDH antisense, 5′-GCCTGCTTCACCACCTTCTTGATG-3′, mVEGF164 sense, GCCAGCACATA GAGAGAATGAGC and mVEGGF165 antisense, CAAGGCTCACA GTGATTTTCTGG.

In Vivo Delivery of siRNA.

PolyTran™ (PT) and TargeTran™ (TT), were used for local and systemic delivery of siRNAs, through subconjuctival and tail vein infection, respectively. PT is a class of cationic polypeptides that transformed cells at high efficiency by the positively charged particle surface. TT belongs to ligand-targeted nanoplex with greatly reduced nonspecific interaction with unwanted biomolecules and cells. TT consists of three functional layers: a RGD ligand of peptide H-ACRGDMFGCA-OH that was structurally similar to the previously reported RGD-containing peptides, a PEG steric layer, and a cationic PEI core that concentrate siRNA or other macromolecules (FIG. 1). By design, the TT-siRNA formulation targets the angiogenic tissue where the RGD-specific intergrins are up-regulated. The efficacy of TT-siRNA formulation has been proven in a previous study in a xenograft breast cancer mouse tumor model where the delivery of siRNAs targeting human VEGF and mouse VEGFR2 achieved substantial inhibitory effect on tumor growth.

Evaluation of Anti-angiogenic Efficacy of siRNA in vivo.

The efficacy of siRNA in inhibition of cornea NV and HSK was evaluated through three ways.

1) Measurement of angiogenic area: On day 4 and 7 after CpG ODN pellet implantation, the length and width of NV area were measured using calipers under a stereomicroscope. The neovessels, originating from the limbal vessel ring toward the center of the cornea, were presented in clock hours where each clock hour equals to 1/12 of the circumstance. The NV area was calculated according to the formula for an ellipse. A=[(clock hours)×0.4 (vessel length in mm)]/2.

2) Clinical scoring of the HSK severity: Eyes were examined on different days after HSV infection for the development of clinical lesions using a split-lamp. The clinical severity of keratitis lesion was scored by the following system: 0, normal cornea; +1, mild corneal haze; +2, moderate corneal opacity or scarring; +3, severe corneal opacity but iris visible; +4, opaque cornea and corneal ulcer; +5, corneal rupture and necrotizing stromal keratitis.

3) Clinical scoring of the NV severity: The severity of angiogenesis was recorded as described previously. Briefly, a grade of 4 for a given quadrant of the circle represents a centripetal growth of 1.5 mm towards the corneal center. The score of the four quadrants of the eye were then summed to derive the neovascularization index (range 0-16) for each eye at a given time point.

xx. RNA template-specific RT-PCR

Measurement of siRNA Sequences for Three Murine VEGF Pathway Genes In Vitro.

To evaluate gene inhibition by the candidate siRNA sequences, a series of transfections in cell culture was performed and the effect on mRNA levels determined as a measure of siRNA activity. Measurements were performed using RS-PCR and RT-PCR methods, described above. Evidence was obtained for knockdown of all three VEGF pathway genes with the siRNA sequences, shown in FIG. 2. In some cases the inhibition is of endogenous expression and in other cases of exogenous expression.

Inhibition of mVEGF-A In Vitro.

The ability of siRNA sequences to inhibit VEGF-A was evaluated in RAW264.7 NO (−) cells, a mouse macrophage cell line that expresses VEGF-A endogenously, shown in FIG. 2A. Expression of both the 120 and 164 isoforms of mVEGF-A, as measured by RT-PCR, VEGF-A expression was reduced in a dose-dependent manner, while transfection of control siRNA had no effect.

Inhibition of mVEGF-R1 in vitro.

The activity of siRNA sequences to inhibit VEGF R1 was evaluated in SVR cells that endogenously express this VEGF receptor. As shown in FIG. 2B, mVEGFR1 expression 48 h post transfection, as measured by RS-PCR, was diminished in a dose dependent manner, while transfection of control siRNA had no effect on the mVEGFR1 level.

Inhibition of mVEGF-R2 in vitro.

Finally, the activity of siRNA sequences to inhibit VEGF R2 was evaluated in 293 cells using a co-transfection approach for exogenous expression by the plasmid pCImEGFR2. FIG. 2C shows that mVEGFR2 expression was reduced, while co-transfection of control siRNA had no effect.

These in vitro study results indicate that expression of all three murine VEGF-pathway genes studied can be inhibited by the siRNA sequences identified with bioinformatics. Based upon this achievement, it was appropriate to evaluate in vivo delivery and activity of siRNA in ocular tissues.

Delivery of Fluorescent siRNA into Murine Ocular Tissues

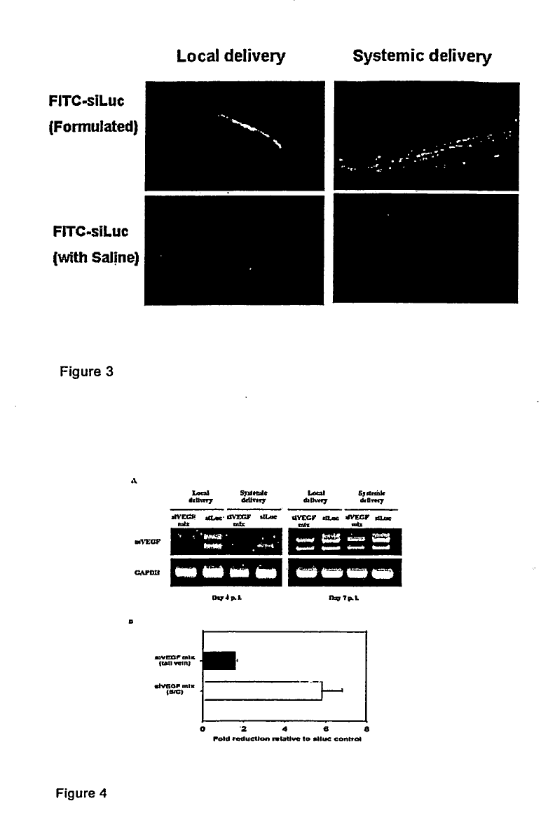

Studies were performed where FITC-labeled siRNA was administered into ocular tissues and the tissues removed to visualize distribution of the siRNA. The results of local administration are shown in FIG. 3A (left panel). The results demonstrated that incorporation of the siRNA into a PolyTran™ cationic peptide polymer facilitated uptake into corneal tissues (upper left panel), while fluorescence from aqueous (non-formulated) siRNA was rapidly lost (lower left panel).

VEGF siRNA Gene Inhibition in Murine Ocular Neovascular Tissues

Studies were performed to determine if local administration of VEGF siRNA inhibited expression levels of VEGF mRNA. Mice cornea were infected with HSV to induce ocular neovascularization and treated with siRNA for mVEGF-A gene by local administration was monitored an example to check the possible changes at RNA or protein level. Corneas were collected at day 4 or 7 from mice that were infected with 1×10⁵ pfu HSV-1 and treated with siRNA on day 1 and day 3. The mVEGF-A mRNA level was measured by RT-PCR or QRT-PCR. The expression of mVEGF-A mRNA was reduced in the cornea treated with siVEGFmix compared with unrelated siLuc control on day 4 and 7 as detected by RT-PCR (FIG. 4A). Similarly, cornea treated with VEGF siRNA showed significant reduction in mVEGF-A mRNA expression in comparison to cornea treated with control siRNA sequence on day 7 detected by QRT-PCR (FIG. 4B), where systemic delivery exhibited stronger efficacy that delivering locally.

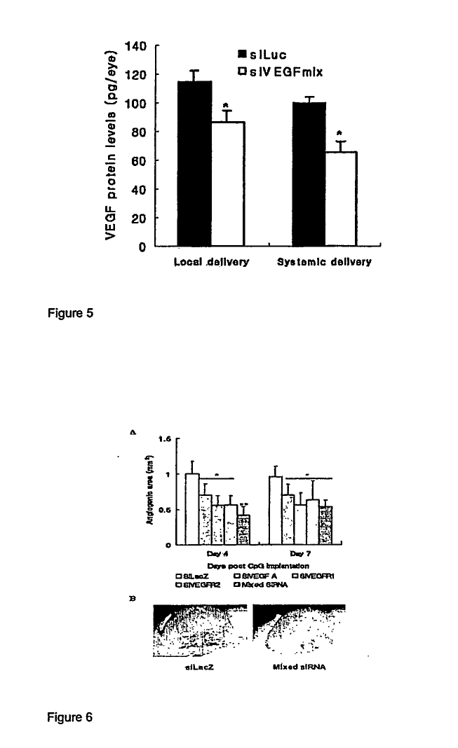

The treatment of HSV infected mice with siRNA also showed inhibition of mVEGF-A by measurement of protein levels on day 7, p.i., as monitored by ELISA, compared with treatment with control siRNA (p<0.05) (FIG. 5). The observed reduction of mVEGF-A expression at RNA or protein level confirmed that the antigenic effect of siRNA on HSV-induced NV and HSK (to be described later in the text) were related to the siRNA-mediated knockdown of the targeted VEGF-pathway factors.

Local Treatment of CpG Induced Corneal Neovascularization with siRNA

Implantation of CpG-containing ODN in hydron pellets into murine ocular tissue (corneal micropockets) induces VEGF-mediated angiogenesis much like HSV infection mediated HSK. This model system avoids handling HSV. This system was employed for measuring the inhibitory effect of local administration of siRNA carried by the polymer carrier, PolyTran™. All the three pairs of siRNA with activity in vitro (FIG. 2) were studied, targeting individually mVEGF-A, mVEGFR1 and mVEGF R2. A single dose of 10 μg/10 μl/eye of siRNA was administered by subconjunctival injection 24 h after the insertion of CpG ODN into micropockets. Each pair of the siRNA duplexes (targeting VEGF-A, VEGF R1, and VEGF R2, respectively) was tested either individually or in combination (at equal molar ratio) at the same total RNA dosage, i.e., 10 μg/10 μl/eye. Neovascularization starting from the corneal limbus (corneal NV) was monitored at both day 4 and 7 after pellet implantation. As shown in FIG. 6, significant inhibition of corneal NV was observed with all three tested siRNA pairs, compared with unrelated siRNA control (LacZ), on day 4 and day 7 after pellet implantation (p<0.05). The combination of the siRNA duplexes targeting all three tested genes demonstrated the most effective inhibition, an approximately 60% reduction in NV (p<0.01) as measured on day 4. The inhibition on day 7 was the most effective. These in vivo results demonstrated the efficacy of the tested siRNA in vivo; suggested the PolyTran™ to be a reasonable choice of siRNA delivery vehicle for local delivery into animal eyes; and also indicated that the potential for combined use of siRNA targeting different genes to achieve synergistic inhibition of pathology.

Systemic Treatment of CpG ODN Induced Angiogenesis HSK Model

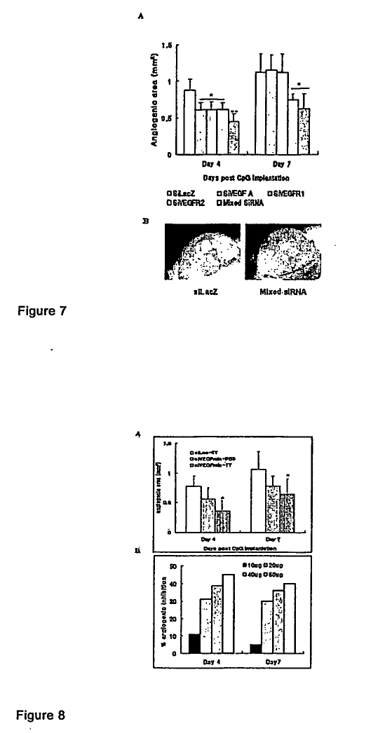

With systemic treatment showing localization of the siRNA at the sites of ocular NV, the therapeutic potential of systemic delivery of siRNA was studied. Mice were given a single dose of 40 μg/100 μl of siRNA targeting mVEGF-A, mVEGFR1, and mVEGFR2, respectively through tail vein injection 24 h post pellet implantation. siRNA were given singly or in combination with same total dosage of siRNA the same way as in above local delivery study. On day 4 and 7 after pellet implantation, the angiogenesis areas were measured as described in the Methods. As shown in FIG. 7, all tested siRNA duplexes demonstrated significant inhibition of NV when used singly, compared with the siLacZ treated group, on day 4 after pellet implantation (p<0.05). Similar to what observed with local administration, the administration of the mix of the siRNAs targeting all three target genes provided the most effective inhibition (approximately 60% inhibition, p<0.01) compared with singly used siRNAs targeting only one target gene. Again, although the efficiency of inhibition on day 7 was generally lower than that on day 4, the mixed siRNA was the most effective compared with singly applied antiangiogenic siRNAs. To confirm that the observed efficacy of TargeTran™-mediated delivery was due to the delivery formulation, we compared the antiangiogenic efficacy of siRNA in the same mouse model when delivered systemically with TargeTran™ or with only PBS. Clearly, TargeTran™-mediated delivery of siRNA achieved more effective antiangiogenesis than those delivered with the PBS vehicle (p<0.05) on day 4 or day 7 post implantation (FIG. 8A). To further confirm the observed efficacy of the TargeTran™-siRNA formulation, a dose response study of antiangiogenic efficacy was also performed in the same CpG-SK mouse model. Mice with CpG ODN-containing micropockets were treated with two doses of tail vein injection of combined siRNA duplexes at 10, 20, 40 and 80 μg dosages at 6 and 24 h post pellet implantation. The antiangiogenic effects evaluated on day 4 and day 7 all indicated a clear dose-dependent pattern (FIG. 8B).

Treatment of HSV Infection-Induced Corneal Neovascularization with siRNA

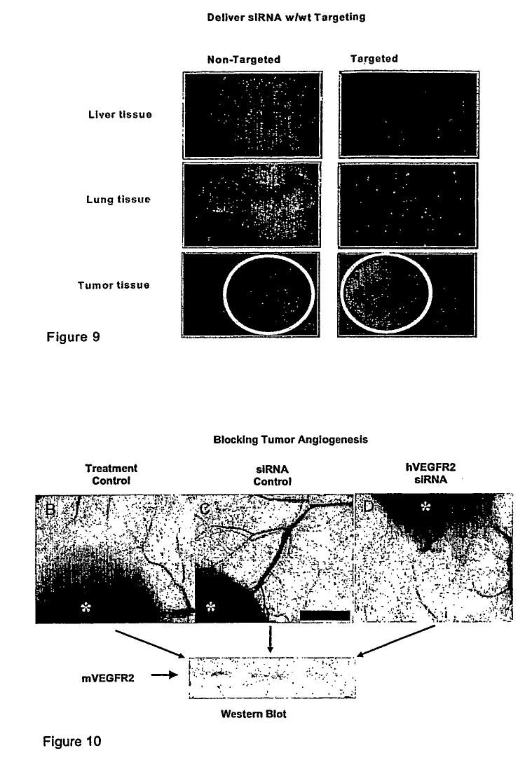

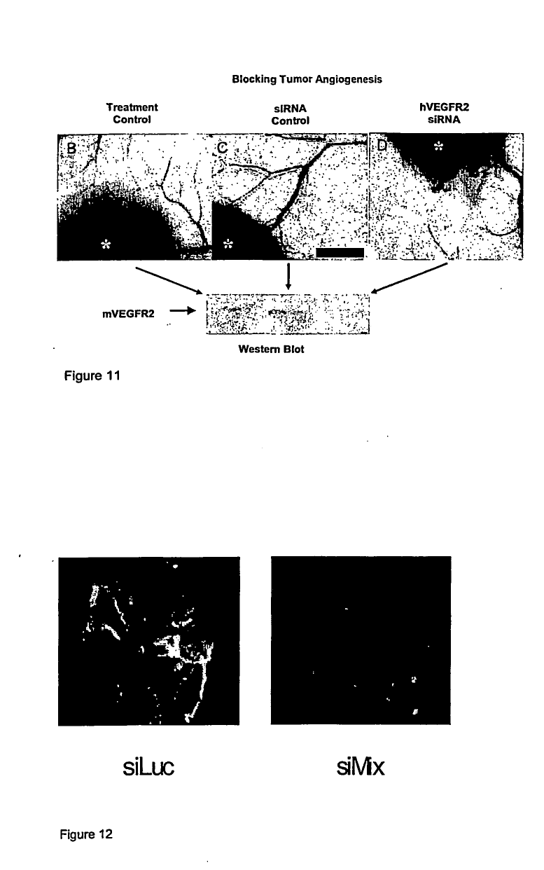

Given the efficacy of VEGF siRNA to inhibit corneal NV in the CpG-ODN model of HSK, studies were performed with the more clinically relevant HSV infection-mediated HSK model. In this study, this system was employed again with local administration of siRNA. Each pair of the siRNA duplexes (targeting VEGF-A, VEGF R1, and VEGF R2, respectively) was tested either individually or in combination (at equal molar ratio) at the same total RNA dose. Both stromal keratitis and neovascularization were monitored over 14 days after HSV infection. As shown in FIG. 9, significant inhibition of both measures (SK and corneal NV) was observed with all three tested siRNA pairs, either locally or systemically, compared with animals treated with Luc siRNA control (p<0.05). Whilst 80% of Luc siRNA control treated eyes developed clinically evident lesions (score 2 or greater at day 10 p.i.), only 42% (local delivery) or 50% (systemic delivery) of eyes treated with siRNA targeting VEGF-pathway genes developed such lesions, where local delivery exhibiting even stronger antiangiogenic efficacy than the systemic delivery. Taken together, these results showed that administration of siRNA against VEGF-pathway genes reduced the development of HSK via inhibition of angiogenesis. As observed with the CpG micropocket model (FIG. 7), the combination of the siRNA duplexes targeting all three VEGF-pathway genes provided the most effective inhibition.

Targeted Delivery of FITC-siRNA into Angiogenic Eyes

To test whether systemic delivery of siRNA with RGD peptide targeted nanoparticles can provide localization at ocular neovascularization, studies were performed by administration of FITC-labeled siRNA with TargeTran™ in the CpG-ODN model of HSK. The results, shown in FIG. 3 (right panel) demonstrate similar uptake by either the local or systemic methods. Also, systemic administration of the FITC-labeled siRNA with the nanoparticle resulted in distribution of fluorescence mainly into the angiogenic eye with much lower levels in liver, kidney, or lung (data not shown). Not surprisingly, systemic administration of a saline siRNA solution failed to show distribution of the siRNA into the angiogenic eyes (FIG. 3, right panel). The observed distribution of FITC-siRNA into the angiogenic eye with subconjunctival injection with PolyTran™ is most likely attributable to the local delivery close to where the new blood vessels are forming (limbus). The ocular neovascular delivery observed with TargeTran™, however, is an expected feature of targeting to integrin expression up regulated at sites of neovascularization for an RGD ligand-targeted nanoplex (FIG. 1).

Cocktail siRNA Application

We repeatedly tested the “cocktail approach” to apply siRNA duplexes in combination. When mixed siRNA targeting different sequences of a same mRNA (in tissue culture, data not shown), or targeting all three tested VEGF-pathway genes (in vivo, FIG. 4-8) was used simultaneously, enhanced potency compared to that achieved when siRNA targeting single gene was used.

Substantial inhibition of corneal NV and SK by siRNA targeting mVEGFA, mVEGFR1, and mVEGFR2, was achieved in the mouse SK model through local or systemic delivery of siRNA. This siRNA-mediated inhibition was achieved either in the CpG-induced corneal NV and SK (FIG. 6, 7, 8) or in that caused by HSV infection (FIGS. 4, 5, and 9) in the tested mice HSK models, in a dose-dependent manner (FIG. 8). This inhibitory effect was compatible with the siRNA-mediated in vitro and in vivo knockdown of targeted mRNAs detected by RS-PCR, RT-PCR, and QRT-PCR (FIG. 2, 4), and with the in vivo knockdown of protein expression of the targeted gene(s) detected by ELISA (FIG. 5). It was also observed that the simultaneous administration of siRNA targeting all the three tested VEGF-pathway genes exhibited antiangiogenic effect stronger than that achieved when siRNA were used singly (FIG. 4-8). The observation on the unrelated Luc siRNA or LacZ siRNA controls and saline control in these in vitro or in vivo experiments confirmed that the siRNA targeting VEGF pathway and the delivering reagents were responsible for the observed corneal NV inhibition.

In corneal delivery testing, PolyTran™ and TargeTran™, appeared to provide efficient delivery of FITC-labeled siRNA into the CpG-treated eyes. The PolyTran™-related ocular delivery appeared attributable to the location of delivery, but the TargeTran™-related ocular delivery likely was attributable to the RGD ligand targeting of the nanoplex. Although it is not clear whether the fluorescence detected was from the intact or degraded siRNA molecules, and the half life of the delivered RNA duplex could not be disclosed by fluorescence detection, it is proper to conclude that selective delivery of siRNA into angiogenic eyes was achieved through the TargeTran™-mediated systemic route.

The multiple-targeting approach described above provides improved siRNA-mediated gene knockdown. When mixed siRNA targeting all the three tested VEGF-pathway genes was used simultaneously, either in tissue culture or in mice, the results always showed stronger potency than that achieved when siRNA targeting a single gene was used (FIG. 4-8). This approach, which we term the “cocktail approach”, can be used for the knockdown of multiple target sequences of a single gene, multiple siRNA targeting different genes (or sequences) of a pathway, multiple genes of infectious agents and host genes (e.g., viral protein and host receptor proteins), etc. This approach applies to corneal SK, other angiogenic diseases including tumors, and the principle applies also to other diseases and biological process.

This present example demonstrates that the use of VEGF pathway-specific siRNA with a clinical delivery system is a novel therapeutic for ocular NV diseases including HSV-induced corneal SK, diabetic retinopathy (DR), age-related macular degeneration (AMD), uveitis, and rubeosis. The ligand-targeting non-viral delivering reagents hold advantages in safety, noninvasiveness, and tissue specificity for therapeutic delivery of siRNA and for treatment of ocular NV disease.

### Example 2

### Local Administration of VEGF Pathway Inhibitors to Treat Posterior Ocular NV Disease

Materials and Methods

Mouse pups with a foster mother were subjected to hypoxia (75%) from P7 to P12, and switched to normal air (normoxia) from P12 to P16. (P number refers to day). Subconjunctival administration was used to deliver siRNA complexed with a cationic polymer reagent, PolyTran PT73. The ratio of siRNA to PT73 was 1:8, by weight. A 5 mM HEPES solution was used to dilute the mixture to the required volume. The siRNA dosage was 4 μg siRNA in the PT73 complex dispersed in a volume of 5 μl, per eye. One injection was performed on P12 and P13 each. For each mouse, the left eye was treated with a negative control siRNA, siLuc, and the right eye was treated with the active siRNA, siMix. The negative control siluc was an equal mixture of two oligos (siLuc-a & b), and the siMix was an equal mixture of simVEGFA, simVEGFR1, and simVEGFR2, each a mixture of two oligos (e.g., simVEGF-a & b, simVEGFR1-a & b, simVEGFR2-a & b). The mice were sacrificed on P16 and fluorescent perfusion/flat mounting and cryosection analyses performed to characterize neovascularization.

Results

The flat mount of two eyes are shown in FIG. 5. One flat mount result shows strong fluorescence that results from perfusion of the dye when extensive neovascularization has occurred, that of the eye treated with siLuc. The other flat mount result shows substantially less fluorescence, an indication of an inhibition of neovascularization in the eye treated with siMix.

### Example 3

### Distal, Systemic Administration of VEGF Pathway Inhibitors to Treat Neovascularization using Tumor Model System

Materials and Methods

Nucleic Acids

Short double stranded RNA oligonucleotides for siRNA labeled siLuc, siLacZ, siGFP, and siVEGFR2 were designed based on studies by Elbashir et al. (2), validated to lack significant interfering homology by BLAST-analysis, and synthesized and purified by Dharmacon (Lafayette, Colo.). Two sequences were synthesized per target and combined in a 1:1 molar ratio. Target sequences used were for siLuc: aaccgctggagagcaactgca and aagctatgaaacgatatgggc, for siLacZ: aacagttgcgcagcctgaatg and aacttaatcgccttgcagcac, for siGFP: aagctgaccctgaagttcatc and aagcagcacgacttcttcaag and for siVEGFR2: aatgcggcggtggtgacagta and aagctcagcacacagaaagac (inhibition of VEGF R2 by this siRNA has been described (28)). siRNA targeted against luciferase was labeled with fluorescein (FITC-siRNA) at the 3′ position of the sense strand with standard linkage chemical conjugation, for FACS-analysis and tissue distribution experiments. The luciferase-encoding pCI-Luc plasmid (pLuc) was obtained from Lofstrand Labs (Gaithersburg, Md.).

Synthesis of RGD-PEG-PEI (RPP) and PEG-PEI (PP)

Two PEGylated forms of branched PEI (P) were prepared, one with a PEG having an RGD peptide at its distal end (RPP) and the other with a PEG lacking the peptide (PP). The abbreviations for these three compounds used here are P for branched PEI, PP for PEGylated PEI, and RPP for RGD-PEG-PEI.

The cyclic 10-mer RGD-peptide with the sequence H-ACRGDMFGCA-OH, was synthesized, oxidized to form an intramolecular disulfide bridge and purified to >95% purity by Advanced ChemTech (Louisville, Ky., USA). This sequence was derived from the integrin binding RGD peptides identified by phage display and has been found effective for cell binding and internalization (23,29).

Synthesis of RPP was carried out as follows in two steps. In the first step, to a stirred solution of RGD (60 mg) in DMSO (600 μL) was added TEA (8.54 μL in 20 μL of TBF) under nitrogen. After stirring for 1 min, a solution of NHS-PEG-VS (212 mg in THF: DMSO; 300 μL: 100 μL) was added in one portion. The reaction mixture was stirred at room temperature for 4 h, quenched with TFA (amount equivalent to the TEA) and the mixture was lyophilized. The intermediate RGD-PEG-VS was purified by either reverse phase BPLC or dialysis against water, and the compound lyophilized to give a yield of 50-90%. Conjugation was confirmed by Mass Spectral analysis (MALDI).

In the second step of synthesis, 100 mg (21.7 μmole) of the purified RGD-PEG-VS intermediate was dissolved in 1 ml of pure DMSO. To this solution 6 equivalents of TEA dissolved in 0.5 ml THF was added and mixed. 9.4 mg (218 μmole in terms of amines) of PEI dissolved in DMF (0.5 ml) was added to the above solution and stirred at room temperature for 12 hours. The completion of the conjugation was confirmed by disappearance of RGD-PEG-VS on TLC. The reaction was terminated by addition of an excess of TFA and lyophilized. The product was purified as the TFA salt by HPLC. Degree of conjugation of RGD-PEG to PEI was determined by proton NMR spectrometry on a 500 MHz spectrometer (Varian), from the ratio of the area under the peaks corresponding to the —CH2— protons of PEI (2.8-3.1 ppm) and PEG (3.3-3.6 ppm). Based on this estimate, ˜7% of the PEI amines were conjugated with RGD-PEG, or an average of about 40 RGD-PEG molecules attached to each 25 KD PEI molecule reducing the average number of amines from 580 to 540 per PEI molecule. Percentage conjugation ranged from 7 to 9 for various syntheses.

Preparation of Nanoplexes

Nanoplexes were prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in formation of polyplexes with average particle size distribution of about 100 nm, and are referred to here as nanoplexes.

Three forms of nanoplexes were prepared based on the three forms of PEI: branched PEI (P), PEGylated PEI (PP) and RGD-PEG-PEI (RPP). Earlier studies have revealed that conjugation of polycations used for DNA condensation with other macromolecules can lead to incomplete condensation and formation of structures with non-spherical morphology (30,31). Though we have not observed any of these problems with the conjugates used in this study, in order to avoid this potential problem, part of the polycation required for condensation was substituted with un-conjugated PEI. Therefore all RPP and PP nanoplexes contain PEI in molar equivalent to the conjugates, expressed in terms of amine concentration. These nanoplexes were thus prepared by first preparing an aqueous solution of cationic polymer containing RGD-PEG-PEI (RPP) or PEI-PEG (PP) with PEI (P) in a 1:1 molar ratio in 5 mM Hepes buffer (pH 7). In a separate tube, nucleic acids (plasmid DNA and/or siRNA) were dissolved in the same buffer in the same total volume as the cationic polymer solution. The two solutions were then mixed together and vortexed for 30 seconds to make nanoplexes. Mean particle size distribution was determined with a Coulter N4plus particle size instrument (Beckman Coulter), and ζ-potential measurements were performed on a Coulter Delsa 440 SX instrument. Both instruments were calibrated using latex beads of defined size and mobility as standards (Beckman Coulter, Miami, Fla.).

Mouse Tumor Neovascularization Model

Female nude mice (6-8 weeks of age) were obtained from Taconic (Germantown, N.Y.), kept in filter-topped cages with standard rodent chow and water available ad libitum, and a 12 h light/dark cycle. Experiments were performed according to national regulations and approved by the local animal experiments ethical committee. Subcutaneous N2A tumors were induced by inoculation of 1×106 N2A-cells in the flank of the mice. At a tumor volume of approximately 0.5-1 cm3 the tumors begin to exhibit neovascularization and at this point mice received nanoplexes or free siRNA by i.v. injection of a solution of 0.2 ml via the tail vein. For tissue distribution experiments, 40 μg fluorescently-labeled siRNA was injected in the free form or as P- or RPP-nanoplexes. One hr after injection, tissues were dissected and examined with a dissection microscope fitted for fluorescence. Microscopic examination of tissues was performed with an Olympus SZX12 fluorescence microscope equipped with digital camera and connected to a PC running MagnaFire 2.0 camera software (Optronics, Goleta, Calif.). Pictures were taken at equal exposure times for each tissue.

In the tumor neovascularization phenotype studies, the experiment was started when the tumors became palpable, at 7 days after inoculation of the tumor cells. Treatment consisted of 40 μg siRNA per mouse in RPP-nanoplexes every 3 days intravenously via the tail vein. Tumor growth was measured at regular intervals using a digital caliper by an observer blinded to treatment allocation. Each measurement consisted of tumor diameter in two directions approximately 90 degrees apart. Tumor volume was calculated as, 0.52×longest diameter×shortest diameter2 (32). At the end of the experiment, the animals were sacrificed and tumor tissue and surrounding skin was excised and put on a microscopy glass slide to determine neovascularization. Tissue examination for neovascularization and angiogenesis was performed by microscopy using the Olympus microscope and camera equipment described above for fluorescent tissue measurements. Tissue was trans-illuminated to visualize blood vessels in the skin and a digital image was taken and stored as described above. Tissue was snap frozen immediately thereafter for Western blotting.

Western Blotting

Murine VEGF-receptor 2 expression in tumor samples was detected by Western blotting. Tumor tissue was put into lysing Matrix D (Bio-Rad, Cambridge, Mass.) together with M-Per mammalian protein extraction reagent (Pierce). Tissue was homogenized, centrifuged and supernatants collected. Equivalent amounts of extracted protein (50 μg) were mixed with sample buffer containing 5% 2-mercaptoethanol (Bio-Rad), boiled, cooled and loaded in each lane of a 6% polyacrylamide gel. Electrophoresis was performed at 30 mA and subsequently proteins were transferred to an Immunblot PVDF membrane (Bio-Rad). Membranes were blocked overnight with 3% gelatin in Tris-buffered saline (TBS). Subsequently, membranes were transferred to 1% gelatin in TTBS (10 mM Tris-HCL, 150 mM NaCl, 0.1% Tween 20) incubated with 1 μg monoclonal anti-mVEGFR2 antibody (R&D Systems) overnight. After washing in TTBS twice, goat anti-mouse-IgG-peroxidase conjugate was added in 1% gelatin for 1 h, the membrane was washed twice with TTBS and subsequently once with TBS. Antibody was stained using a Bio-Rad color reagent kit for 30 min.

Results

siRNA Nanoplex Colloidal Properties

The siRNA nanoplex was developed with a modular approach to design molecular conjugates that incorporate the three functional requirements: self-assembly, formation of a steric polymer protective surface layer, and exposed ligands. To design an siRNA nanoplex, we revisited materials used originally for plasmid DNA including polyethyleneimine (PEI) for the polycation complexing agent, polyethylene glycol (PEG) for steric stabilization, and peptide ligands containing an Arg-Gly-Asp (RGD)-motif to provide tumor selectivity due to their ability to target integrins expressed on activated endothelial cells in tumor vasculature. While peptides containing an RGD motif can bind several integrins, their specificity is determined by the flanking amino acid sequence as well as the conformation of the binding domain. In this study, we used a “cyclic” RGD peptide whose integrin binding domain is conformationally constrained by a disulfide bond. It has an identical amino acid sequence within the cyclic region of a peptide that was shown to cause cell binding and internalization by a receptor mediated pathway when expressed on filamentous phage. This peptide was shown to inhibit cell attachment to fibronectin and vitronectin coated plates in a sequence specific manner. Further, when coupled to an oligo-lysine this peptide showed receptor mediated DNA delivery in a variety of cells including endothelial cells. In order to facilitate chemical conjugation to PEG, for these studies an alanine residue was added to each end of this peptide outside the cyclic region. The targeted siRNA nanoplexes were prepared by chemical synthesis of tripartite polymer conjugates with a cationic polymer, a steric polymer, and a peptide ligand (RPP) followed by nanoparticle self-assembly by mixing with nucleic acid in aqueous solution. This RPP conjugate allows individualized optimization or chemical replacement for each functional domain.

Upon mixing purified RPP with an aqueous nucleic acid solution, the cationic domain of the conjugate binds to negatively charged nucleic acid, driving self-assembly to form a nanoparticle dispersion. Studies found that stable nanoplexes could be formed at an amine (PEI) to phosphate (nucleic acid) ratio (N/P) of 2:1. Particle size and zeta potential results at this ratio are given in Table I. The mean size of either RPP- or PP-nanoplexes was small, between 0.07 and 0.10 μm. Particle size remained largely unchanged for 9 days, a period for which particle size was monitored. In contrast, the mean particle size of P-nanoplexes was larger, between 0.12 and 0.17 μm, and aggregated within 24 hours. The ζ-potential of P-nanoplexes with siRNA was found to be highly positive, 35±4 mV, as typically found with plasmid DNA, but incorporation of the PEG-conjugate of PEI resulted in a reduction in the ζ-potential to 5±6 mV and 6±1 mV for PP-nanoplexes and RPP-nanoplexes, respectively. Surface charge polarity and amplitude depended on the ratio of the two components but at the same ratio amplitude decreased when PEG was present (data not shown), indicating that a steric polymer layer is formed on the nanoplex surface.

These measurements of particle size and zeta potential indicate that the RPP nanoplexes formed with siRNA exhibit colloidal surface properties indicative of an outer steric polymer layer and potentially exposed RGD ligand to mediate cell binding selectivity. This nanoplex self-assembly occurs by simple mixing of aqueous solutions of RPP conjugates with siRNA (RPP-nanoplexes). Their colloidal and biological properties were compared either to preparations with precursor conjugates lacking the RGD peptide (PP-nanoplexes) or to unconjugated PEI (P-nanoplexes).

Tumor Uptake, Targeted Gene Inhibition, Phenotypic Effect

RPP-nanoplexes were selected for in vivo studies on inhibition of neovascularization in tumor-bearing mice. Studies were performed to determine increased tumor neovascularization levels of siRNA by siRNA nanoplexes administered by iv injection to tumor-bearing animals. Imaging of FITC-labeled siRNA uptake into established neuroblastoma N2A-tumors in nude mice was used to observe tumor neovascularization accumulation. Fluorescence microscopy to detect FITC fluorescence in tumor, lung and liver are shown in Figure for animals administered aqueous siRNA, P-nanoplexes, and RPP-nanoplexes. Intravenous administration of aqueous siRNA did not produce appreciable FITC-siRNA fluorescence in the tumor. Also, for this sample very little FITC fluorescence can be observed in the liver and even less in the lung. These results are most likely a reflection of rapid clearance of the FITC-siRNA into the urine, poor tissue accumulation except liver, and potentially metabolic instability resulting in rapid excretion or liver metabolism of the FITC. The lack of tumor fluorescence from the aqueous FITC-siRNA indicates that any instability of the FITC linkage that would result in loss of FITC from nanoplex preparations will not yield tumor fluorescence. This conclusion is confirmed by a lack of tumor FITC fluorescence when P-nanoplexes were administered. This lack of tumor FITC fluorescence demonstrates that neither the P-nanoplexes accumulate in the tumor to any detectable extent nor does any FITC linkage stability in the siRNA nanoplex result in artifactual tumor fluorescence. On the other hand, the FITC-siRNA in P-nanoplexes does produce appreciable FITC-siRNA fluorescence in liver and especially lung with a punctate profile. In contrast, RPP-nanoplexes produced appreciable FITC-siRNA fluorescence in the tumor neovasculature, but poor liver and lung accumulation and a reduced punctate fluorescence pattern. This provides strong evidence that the RPP-nanoplex exhibits reduced nonspecific tissue interactions reducing the liver and lung uptake and accumulation in tumor neovascularture due to targeting. Since the FITC fluorescent label is covalently attached to the siRNA with a linkage routinely used for oligonucleotides with known in vivo stability, the fluorescence distribution observed in these tissues corresponds to distribution of siRNA and not to FITC linkage instability. Furthermore, even administration of the FITC conjugated siRNA in aqueous form, which is more sensitive to degradation in serum, did not result in any significant accumulation in any of the tissues measured. These results indicate that the RPP siRNA nanoplex gives increased levels at tumor neovasculature of siRNA molecules and led to studies of siRNA biological activity in the tumor described below.

Whether the siRNA inhibited neovascularization through RPP mediated delivery into tumor was determined using an siRNA targeted to an endogenous therapeutic gene. For these studies, siRNA targeting murine vascular endothelial growth factor receptor-2 (VEGF R2) was selected and used with RPP nanoplexes since it is a pivotal factor in angiogenesis. For therapeutic effects on this gene, though, the siRNA requires delivery into host (murine) endothelial cells within the tumor to elicit a phenotypic effect on tumor growth. Efficacy studies were performed with siRNA inhibiting expression of murine VEGF R2, characterized in cell culture (data not shown). Studies were performed with this therapeutic gene siRNA administered intravenously as RPP-nanoplexes every 3 days. The results, shown in Figure, show strong inhibition of tumor VEGF R2 levels, neovascularization, and growth rate, and this was sequence specific. Tumor angiogenesis was characterized along with VEGF R2 expression levels. Reduced tumor growth rate was paralleled by reduction in blood vessels in the vicinity immediately surrounding the tumor. Additionally, the few blood vessels visible in the tumor treated with mVEGF R2 siRNA exhibit evidence of erratic branching expected from silencing VEGFR2-expression. Expression of VEGFR2 in treated tumors was also reduced in a sequence specific manner (FIG. 6E). Taken together, these results support the interpretation that the tumor inhibition by siVEGFR2 in RPP-nanoplexes occurs as a result of effective delivery of the siRNA into tumor vasculature producing a sequence-specific inhibition of VEGFR2-expression, tumor angiogenesis and growth. These results show that the siRNA nanoplex acts through a neovascular targeting and inhibition mechanism.

### Example 4

### Distal, Systemic Administration of VEGF Pathway Inhibitors to Treat Posterior Ocular NV Disease

Materials and Methods

Mouse pups with a foster mother are subjected to hypoxia (75%) from P7 to P12, and then switched to normal air (normoxia) from P12 to P16. Intravenous administration is used to deliver siRNA complexed with a cationic polymer reagent, TargeTran RPP. The ratio of siRNA to RPP is over the range of 1:2 to 1:8, by weight. A 5 mM HEPES solution is used to dilute the mixture to the required volume. The siRNA dosage is over the range of 10 μg to 100 μg siRNA in the RPP complex dispersed in a volume of 100 μl, per mouse. Administration commences on P12 in some instances, earlier in other instances, and later in yet other instances. Administration is repeated with different schedules ranging from every day for one week to every week. For each study, a group of mice is treated with a negative control siRNA, siLuc, and another group treated with the active siRNA, siMix. The negative control siluc is an equal mixture of two oligos (siLuc-a & b), and the siMix is an equal mixture of simVEGFA, simVEGFR1, and simVEGFR2, each a mixture of two oligos (e.g., simVEGF-a & b, simVEGFR1-a & b, simVEGFR2-a & b). The mice are sacrificed at different times, commencing on P16. Neovascularization is determined by fluorescent perfusion/flat mounting and cryosection analyses.

Results

The flat mount of eyes shows strong fluorescence that results from perfusion of the dye when extensive neovascularization has occurred, when the mice are treated with siLuc. When neovascularization is inhibited, the flat mount result of eyes shows substantially less fluorescence, when mice are treated with siMix.

### RNAi Agents.

siRNA Agents.

The siRNA agents are designed and prepared following methods of the invention and as illustrated in Example 1. The sequences of suitable siRNA agents are shown in Appendix II below.

1) RNA-specific PCR (RS-PCR)

RS-PCR is used to detect the reduction of mRNA synthesis of the targeted genes. RS-PCR included two consecutive reactions: RNA-specific reverse transcription (RT) and PCR. The total cellular RNA transcript was isolated from the transfected mammalian cells or tissues removed from the tested animals, using RNAwiz (Ambion), and treated with DNase in the way described in Promega's T7RiboMAX manual. The DNA-free RNA is used as template for RT reaction to synthesize the first strand of mRNA-specific cDNA molecules. A mRNA-specific primer designed for the RT contained, from 5′ to 3′ direction, a special 30 nt sequence, which is not complementary to the targeted gene coding sequence, followed by a 14 nt sequence complementary to part of the coding sequence at around 400 nt 3′ to the AUG codon. The RT reaction is performed using MuLv retrotranscriptase (Applied Biosystems) in a volume of 20 ul. The reaction is performed at 37° C. for 30′ followed by 42° C. 15′ and then heated at 94° C. for 5′. PCR reaction was performed using a GeneAmp kit (PE Biosystems). Each pair of primers used for PCR included the forward primer, which was complementary to the coding sequence starting at about 10 nt 3′ to the AUG codon, and the reverse primer, which only has the special 30 nt sequence described above. The 50 ul reaction contains 1 ul of 20 um stocks of each of the pair of primers, 5 ul 10> PCRII buffer, 3 ul 25mM MgCl₂, 1 ul 10 mM dNTPs, and 0.5 ul (5u/ul) TaqDNA polymerase. The PCR reaction was performed at 94° C. 2′, then 35 cycles of 94° C. 1′-72° C. 2′ two-step reaction, followed by 72° C. 10′ and soaked at 4° C. The samples of the reaction are detected by agarose gel electrophoresis along with DNA size standards. The density of DNA fragment of reasonable size (around 400 bp) reflects the starting level of target gene-specific mRNAs in the transfected cells. The siRNAs are synthesized by Dharmacon and primers synthesized by Elim Biopharmaceuticals.

2) Design of the RS-PCR Primers

For mVEGF-A (reference sequence: XM_{—}192823)

Primer 1: mVEGF-A Up (30-mer, 4-33 nt of mVEGF-A coding sequence, or 64-93 nt of cloning sequence).

5′-GAT GTC TAC CAG CGA AGC TAC TGC CGT CCG-3′

Primer 2: mVEGF-A Dn (47-mer, the first 30 is the same as “TS1 primer”, the following 17-mer is complementary to the 403-387 nt of mVEGF-A coding sequence, or 463-447 nt of cloning sequence).

5′-GAA CAT CGA TGA CAA GCT TAG GTA TCG ATA caa gct
gcc tcg cct tg-3′

For mVEGFR-1 (reference sequence: D88689)
Primer 3: mVEGFR-1 Up (30-mer, -33 bp of mVEGFR-1 coding sequence, or 255-284 of cloning sequence)

5′-GTC AGC TGC TGG GAC ACC GCG GTC TTG CCT-3′

Primer 4: mVEGFR-1 Dn (47-mer, the first 30 is the same as “TS1 primer”, the following 17-mer is complementary to the 377-361 nt of mVEGFR-1 coding sequence, or 628-612 nt of cloning sequence).

5′-GAA CAT CGA TGA CAA GCT TAG GTA TCG ATA tag att
gaa gat tcc gc-3′

For mVEGFR-2 (reference sequence: D88689)
Primer 5: mVEGFR2/400Dn (47-mer, 3′ 17-mer complementary to mVEGFR2 400-384 nt)

5′-GAA CAT CGA TGA CAA GCT TAG GTA TCG ATA ggt cac
tga cag agg cg-3′

Primer 6: mVEGFR2/12up (30 -mer, 12-41 of mVEGFR2)

5′-GGC GCT GCT AGC TGT CGC TCT GTG GTT CTG-3′

TABLE 2
RS-PCR targeted genes and the size of products

| # | Target genes | Primers | Size of RS-PCR products |
|---|---|---|---|
| 1 | mVEGF-A | 1&2 | 400 bp |
| 2 | mVEGFR-1 | 3&4 | 374 bp |
| 3 | MVEGFR-2 | 5&6 | 389 bp |

### References:

1. Folkman, J, 1971 N. Engl. J. Med., Vol. 285, p 1182-1186.
2. Leung, D. W. et al. 1989 Science, Vol. 246, p 1306-1309.
3. Aiello, L. P. et al. 1995 PNAS, Vol. 92, pp. 10457-10461.
4. Elbashir, S. M. et al 2001 Genes Dev. 15(2):188-200.
5. McManus, M. T. and Sharp, P. A. 2002: Nature Reviews, Genetics. Vol. 3. p 737-747.
6. Lu, P. Y. et al 2003 Current Opinion in Molecular Therapeutics, 5(3):225-234.
7. Lu, P. Y. et al. 2005. Trend in Molecualr Medicine, 11 (3). 152-160.
8. Bumseok Kim, et al. 2004. American Journal of Pathology, Vol. 165, No. 6.
9. Schiffelers, R. M. et al. Nucleic Acids Research, 2004, Vol. 32, No. 19 e149.
10. Chi, J.-T. et al 2003 PNAS. 1037853100.
11. Semizarov, D. et al. 2003 PNAS. 1131959100.
12. Hough, S. R. et al. 2003 Nature Biotechnology, 21(7) 731-732.
13. Lu, P. Y. et al. 2002 Cancer Gene Therapy, Vol. 10, Supplement 1, 011.
14. Lu, P. Y. et al. 2001 U.S. patent application No. 60/330,909
15. Xie, F. Y. et al. 2002: U.S. patent application No. 60/401,029
16. Mei Zheng et al., 2002 PNAS, Vol. 99(13).
17. Deshpande S P, et al. Vet Microbiol 2002, 86:17-26.
18. Thomas J, et al. Immunol Res 1997, 16:375-386.
19. Zheng M, et al. J Virol 2001, 75:9828-9835.
20. Zheng M, et al. Am J Pathol 2001, 159:1021-1029.
21. Klinman D M, et al. Proc Natl Acad Sci USA 1996, 93:2879-2883.
22. Krieg A M, et al. Nature 1995, 374:546-549.
23. Zheng M, et al. Proc Natl Acad Sci USA 2002, 99:8944-8949.
24. Klinman D M, et al. Drug News Perspect 2002, 15:358-363.
25. Sioud M: Trends Pharmacol Sci 2004,25:22-28.
26. Verma U N, et al. Clin Cancer Res 2003, 9:1291-1300.
27. Sorensen D R, et al. J Mol Biol 2003, 327:761-766.
28. Sanceau J, et al. J Biol Chem 2003, 278:36537-36546.
29. Tuschl T: Chembiochem 2001, 2:239-245.
30. Tuschl T: Nat Biotechnol 2002, 20:446-448.
31. Babu J S, et al. J Immunol Methods 1993, 165:207-216.
32. Kenyon B M, et al. Invest Ophthalmol Vis Sci 1996, 37:1625-1632.
33. Ramanathan M, et al. Exp Biol Med (Maywood) 2003, 228:697-705.
34. Autiero M, et al. J Thromb Haemost 2003, 1:1356-1370.
35. Bushman F:Mol Ther 2003, 7:9-10.
36. Elbashir S M, et al. Nature 2001, 411:494-498.
37. Sioud M, et al. Biochem Biophys Res Commun 2003, 312:1220-1225.
38. Ruoslahti E, et al. Science 1987, 238:491-497.
39. Godbey W T, et al. J Control Release 1999, 60:149-160.
40. Collo G, et al. J Cell Sci 1999, 112 (Pt 4):569-578.
41. Sudhakar A, et al. Proc Natl Acad Sci USA 2003, 100:4766-4771.
42. Lee S, et al. J Clin Invest 2002, 110:1105-1111.
43. Banerjee K, et al. J Immunol 2004, 172:1237-1245.
   Appendix II. siRNA Targeted Sequences for Ocular Diseases and Anti-Angiogenesis Activities

### SS1.VEGF Pathway

### SS1.1. VEGF-A

VEGF gene: human VEGF, Accession: XM_{—}052681, Gene ID: 14781453, mouse VEGF, Accession: M95200, Gene ID: 202350.
   20 siRNA candidates were selected:

| | | |
|---|---|---|
| # | Position | Sequence |

| | | |
|---|---|---|
| VEGF-A-1 | 64-84 | AAGTGGTCCCAGGCTGCACCC |
| VEGF-A-2 | 467-487 | AAGATCCGCAGACGTGTAAAT |
| VEGF-A-3 | 498-518 | AAACACAGACTCGCGTTGCAA |
| VEGF-A-4 | 499-519 | AACACAGACTCGCGTTGCAAG |
| VEGF-A-5 | 517-537 | AAGGCGAGGCAGCTTGAGTTA |
| VEGF-A-6 | 537-557 | AAACGAACGTACTTGCAGATG |
| VEGF-A-7 | 538-558 | AACGAACGTACTTGCAGATGT |
| VEGF-A-8 | 542-564 | AACGTACTTGCAGATGTGACA |
| VEGF-A-9 | 162-182 | AATCGAGACCCTGGTGGACAT |
| VEGF-A-10 | 338-358 | AAGGCCAGCACATAGGAGAGA |
| VEGF-A-11 | 92-112 | AAGGAGGAGGGCAGAATCATC |
| VEGF-A-12 | 386-406 | AATGCAGACCAAAGAAAGATA |
| VEGF-A-13 | 380-400 | AATGTGAATGCAGACCAAAGA |
| VEGF-A-14 | 301-321 | AACATCACCATGCAGATTATG |
| VEGF-A-15 | 451-471 | AAGCATTTGTTTGTACAAGAT |
| VEGF-A-16 | 116-136 | AAGTGGTGAAGTTCATGGATG |
| VEGF-A-17 | 401-421 | AAGATAGAGCAAGACAAGAAA |
| VEGF-A-18 | 421-441 | AATCCCTGTGGGCCTTGCTCA |
| VEGF-A-19 | 379-499 | AAATGTGAATGCAGACCAAAG |
| VEGF-A-20 | 262-282 | AATGACGAGGGCCTGGAGTGT |

### SS1.2. VEGF-B

VEGF-B gene: human VEGF-B, Accession: NM_{—}003377.3, Gene ID: 39725673
   10 siRNA candidates were selected:

| | | |
|---|---|---|
| # | Position | Sequence |

| | | |
|---|---|---|
| VEGF-B-1 | 140-160 | AAAGTGGTGTCATGGATAGAT |
| VEGF-B-2 | 141-163 | AAGTGGTGTCATGGATAGATG |
| VEGF-B-3 | 236-258 | AAACAGCTGGTGCCCAGCTGC |
| VEGF-B-4 | 327-349 | AAGTCCGGATGCAGATCCTCA |
| VEGF-B-5 | 390-412 | AAGAACACAGCCAGTGTGAAT |
| VEGF-B-6 | 393-415 | AACACAGCCAGTGTGAATGCA |
| VEGF-B-7 | 424-446 | AAAGGACAGTGCTGTGAAGCC |
| VEGF-B-8 | 425-447 | AAGGACAGTGCTGTGAAGCCA |
| VEGF-B-9 | 440-462 | AAGCCAGACAGGGCTGCCACT |
| VEGF-B-10 | 670-692 | AACCCAGACACCTGCAGGTGC |

### SS1.3.

VEGF R-1 gene: human VEGF-R1, (hFLT-1), Accession: AF063657, Gene ID: 3132830, mouse VEGF-R1, (mFLT-1), Accession: D88689, Gene ID: 2809068),
   20 siRNA candidates were selected:

| | | |
|---|---|---|
| # | Position | Sequence |

| | | |
|---|---|---|
| VEGFR1-1 | 1706-1728 | AAGGAGAGGACCTGAAACTGT |
| VEGFR1-2 | 2698-2720 | AAGCAAGGAGGGCCTCTGATG |
| VEGFR1-3 | 2702-2724 | AAGGAGGGCCTCTGATGGTGA |
| VEGFR1-4 | 2755-2777 | AACTACCTCAAGAGCAAACGT |
| VEGFR1-5 | 3014-3036 | AAGTGGCCAGAGGCATGGAGT |
| VEGFR1-6 | 3048-3070 | AAAGTGCATTCATCGGGACCT |
| VEGFR1-7 | 3049-3071 | AAGTGCATTCATCGGGACCTG |
| VEGFR1-8 | 2140-2160 | AGCACGCTGTTTATTGAAAGA |
| VEGFR1-9 | 568-588 | AAGGGCTTCATCATATCAAAT |
| VEGFR1-10 | 215-235 | AAAGGCTGAGCATAACTAAAT |
| VEGFR1-11 | 2352-2372 | AAGGTCTTCTTCTGAAATAAA |
| VEGFR1-12 | 3517-3537 | AATGCCATACTGACAGGAAAT |
| VEGFR1-13 | 1190-1210 | AAGAGGATGCAGGGAATTATA |
| VEGFR1-14 | 834-854 | AAGGCGACGAATTGACCAAAG |
| VEGFR1-15 | 89-109 | AAGATCCTGAACTGAGTTTAA |
| VEGFR1-16 | 216-236 | AAGGCTGAGCATAACTAAATC |
| VEGFR1-17 | 3429-3449 | AAGGCCAAGATTTGCAGAACT |
| VEGFR1-18 | 967-987 | AACACCTCAGTGCATATATAT |
| VEGFR1-19 | 567-587 | AAAGGGCTTCATCATATCAAA |
| VEGFR1-20 | 1938-1958 | AATCCTCCAGAAGAAAGAAAT |

### SS1.4.

VEGF R-2 gene: human VEGF-R2, (hKDR), Accession: AF063658, Gene ID: 3132832, mouse VEGF-R2, (mFLK-1), Accession: X70842, Gene ID: 57923 ), 20 siRNA candidates were selected:

| | | |
|---|---|---|
| # | Position | Sequence |

| | | |
|---|---|---|
| VEGFR2-1 | 523-545 | AACAGAATTTCCTGGGACAGC |
| VEGFR2-2 | 2387-2409 | AACTGAAGACAGGCTACTTGT |
| VEGFR2-3 | 2989-3011 | AAGGACTTCCTGACCTTGGAG |
| VEGFR2-4 | 3032-3054 | AAGTGGCTAAGGGCATGGAGT |
| VEGFR2-5 | 3040-3062 | AAGGGCATGGAGTTCTTGGCA |
| VEGFR2-6 | 3401-3423 | AAATGTACCAGACCATGCTGG |
| VEGFR2-7 | 3632-3654 | AATTCCATTATGACAACACAG |
| VEGFR2-8 | 3676-3698 | AACAGTAAGCGAAAGAGCCGG |
| VEGFR2-9 | 3641-3661 | ATGACAACACAGCAGGAATCA |
| VEGFR2-10 | 357-377 | AAGCTCAGCACACAGAAAGAC |
| VEGFR2-11 | 493-513 | AATGCGGCGGTGGTGACAGTA |
| VEGFR2-12 | 1837-1857 | AATGCCACCATGTTCTCTAAT |
| VEGFR2-13 | 2969-2989 | AAGCTCCTGAAGATCTGTATA |
| VEGFR2-14 | 2549-2569 | AAGCAGATGCCTTTGGAATTG |
| VEGFR2-15 | 3906-3926 | AAGCGGCTACCAGTCCGGATA |
| VEGFR2-16 | 2941-2961 | AAGTCCCTCAGTGATGTAGAA |
| VEGFR2-17 | 304-324 | AAGTGCTTCTACCGGGAAACT |
| VEGFR2-18 | 2862-2882 | AATCCCTGTGGATCTGAAACG |
| VEGFR2-19 | 130-150 | AAGGCTAATACAACTCTTCAA |
| VEGFR2-20 | 1204-1224 | AATCCCATTTCAAAGGAGAAG |

### SS2.EGF Pathway

### SS2.1.

EGF gene: Human EGF, Accession: NM_{—}001963, Gene ID: 6031163.
   20 siRNA candidates were selected:

| | | |
|---|---|---|
| # | Position | Sequence |

| | | |
|---|---|---|
| EGF-1 | 2042-2062 | AAGTGGATAGAGAGAGCTAAT |
| EGF-2 | 3873-3893 | AAGGCTGCTGGATTCCAGTAT |
| EGF-3 | 2426-2446 | AAGCAGTCTGTGATTGAAATG |
| EGF-4 | 2621-2641 | AAGCCCTCATCACTGGTTGTG |
| EGF-5 | 1273-1293 | AAAGGACATGGTTAGAATTAA |
| EGF-6 | 2328-2348 | AAGGCCTTGGCCGTCTGGTTA |
| EGF-7 | 174-194 | AAGGGTGTCAGGTATTTCTTA |
| EGF-8 | 3922-3942 | AATGGAGCGAAGCTTTCATAT |
| EGF-9 | 1496-1516 | AAGTACTGTGAAGATGTTAAT |
| EGF-10 | 1274-1294 | AAGGACATGGTTAGAATTAAC |
| EGF-11 | 531-551 | AAGGTACTCTCGCAGGAAATG |
| EGF-12 | 2686-2706 | AAACGGAGGCTGTGAACATAT |
| EGF-13 | 2263-2283 | AATGGCCAAGAGATTATTGTG |
| EGF-14 | 1292-1312 | AACCTCCATTCATCATTTGTA |
| EGF-15 | 261-281 | AAGGTCTCTCAGTTGAAGAAA |
| EGF-16 | 3218-3238 | AATGCCAGCTGCACAAATACA |
| EGF-17 | 1019-1039 | AAGGCTCTGTTGGAGACATCA |
| EGF-18 | 2576-2596 | AAGAGGACTGGCAAAGATAGA |
| EGF-19 | 760-780 | AAGGCAAGAGAGAGTATGTAA |
| EGF-20 | 765-785 | AAGAGAGAGTATGTAATATAG |

### SS2.2.

EGF R gene: Human EGF-R, Accession: NM_{—}005228, Gene ID: 41327737), mouse EGF-R, Accession: NM_{—}207655, Gene ID: 46560581,
   5 siRNA candidates were selected:

| | | |
|---|---|---|
| # | Position | Sequence |

| | | |
|---|---|---|
| EGFR-1 | 483-505 | AAAGACCATCCAGGAGGTGGC |
| EGFR-2 | 2869-2889 | AAAGTGCCTATCAAGTGGATG |
| EGFR-3 | 2870-2890 | AAGTGCCTATCAAGTGGATGG |
| EGFR-4 | 3751-3771 | AACCCTGACTACCAGCAGGAC |
| EGFR-5 | 3755-3775 | CTGACTACCAGCAGGACTTCT |

### SS2.3.

HER-2 gene: Human HER-2, Accession: M11730, Gene ID:183986, mouse HER-2, Accession: BC053078, Gene ID: 31419374,
   5 siRNA candidates were selected:

| | | |
|---|---|---|
| # | Position | Sequence |

| | | |
|---|---|---|
| HER2-1 | 1255-1275 | AAGATCTTTGGGAGCCTGGCA |
| HER2-2 | 1253-1273 | AAGAAGATCTTTGGGAGCCTG |
| HER2-3 | 2797-2817 | AAGGTGCCCATCAAGTGGATG |
| HER2-4 | 3019-3039 | AAATGTTGGATGATTGACTCT |
| HER2-5 | 3805-3825 | AACCTCTATTACTGGGACCAG |

### SS2.4.

HER-3 gene: Human HER-3, Accession: M34309, Gene ID:183990, mouse HER-3, Accession: XM_{—}125954, Gene ID: 38091004,
   13 siRNA candidates were selected:

| | | |
|---|---|---|
| # | Position | Sequence |

| | | |
|---|---|---|
| HER3-1 | 678-698 | AATTGACTGGAGGGACATCGT |
| HER3-2 | 1264-1284 | AAGATCCTGGGCAACCTGGAC |
| HER3-3 | 1537-1557 | AAGGAAATTAGTGCTGGGCGT |
| HER3-4 | 2404-2424 | AAGATTCCAGTCTGCATTAAA |
| HER3-5 | 2857-2877 | AAATACACACACCAGAGTGAT |
| HER3-6 | 2858-2878 | AATACACACACCAGAGTGATG |
| HER3-7 | 3770-3790 | AAGATGAAGATGAGGAGTATG |
| HER3-8 | 3776-3796 | AACCTCTATTACTGGGACCAG |
| HBR3-9 | 1118-1138 | CTGACAAGATGGAAGTAGATA |
| HER3-10 | 1119-1139 | TGACAAGATGGAAGTAGATAA |
| HER3-11 | 2402-2422 | TCAAGATTCCAGTCTGCATTA |
| HER3-12 | 2403-2423 | CAAGATTCCAGTCTGCATTAA |
| HER3-13 | 2805-2825 | TGAGGCCAAGACTCCAATTAA |

### SS2.5.

HER-4 gene: Human HER4, Accession: NM_{—}005235, Gene ID:4885214, mouse HERA, Accession: XM_{—}136682, Gene ID: 38049556.
   7 siRNA candidates were selected:

| | | |
|---|---|---|
| # | Position | Sequence |

| | | |
|---|---|---|
| HER4-1 | 462-482 | AAATGGTGGAGTCTATGTAGA |
| HER4-2 | 463-483 | AATGGTGGAGTCTATGTAGAC |
| HER4-3 | 731-751 | AATGTGCTGGAGGCTGCTCAG |
| HER4-4 | 838-860 | AATCCAACCACCTTTCAACTG |
| HER4-5 | 1227-1247 | AACAGGTTTCCTGAACATACA |
| HER4-6 | 1450-1470 | AACTGGACAACACTCTTCAGC |
| HER4-7 | 1909-1929 | AACGGTCCCACTAGTCATGAC |

### SS3. FGF Pathway

### SS3.1.

FGF-2 gene: Human FGF-2 (basic FGF), Accession: NM_{—}002006, Gene ID: 41352694.
   20 siRNA candidates were selected:

| | | |
|---|---|---|
| # | Position | Sequence |

| | | |
|---|---|---|
| FGF-2-1 | 630-650 | AAGAGCGACCCTCACATCAAG |
| FGF-2-2 | 661-681 | AAGCAGAAGAGAGAGGAGTTG |
| FGF-2-3 | 849-869 | AAACGAACTGGGCAGTATAAA |
| FGF-2-4 | 880-900 | AAACAGGACCTGGGCAGAAAG |
| FGF-2-5 | 854-874 | AACTGGGCAGTATAAACTTGG |
| FGF-2-6 | 648-668 | AAGCTACAACTTCAAGCAGAA |
| FGF-2-7 | 850-870 | AACGAACTGGGCAGTATAAAC |
| FGF-2-8 | 881-901 | AACAGGACCTGGGCAGAAAGC |
| FGF-2-9 | 667-687 | AAGAGAGAGGAGTTGTGTCTA |
| FGF-2-10 | 723-743 | AAGGAAGATGGAAGATTACTG |
| FGF-2-11 | 734-754 | AAGATTACTGGCTTCTAAATG |
| FGF-2-12 | 781-801 | AACGATTGGAATCTAATAACT |
| FGF-2-13 | 690-710 | AAAGGAGTGTGTGCTAACCGT |
| FGF-2-14 | 818-838 | AAGGAAATACACCAGTTGGTA |
| FGF-2-15 | 804-824 | AATACTTACCGGTCAAGGAAA |
| FGF-2-16 | 750-770 | AAATGTGTTACGGATGAGTGT |
| FGF-2-17 | 822-842 | AAATACACCAGTTGGTATGTG |
| FGF-2-18 | 655-675 | AACTTCAAGCAGAAGAGAGAG |
| FGF-2-19 | 823-843 | AATACACCAGTTGGTATGTGG |
| FGF-2-20 | 798-818 | AACTACAATACTTACCGGTCA |

### SS3.2.

FGF-1 gene: Human FGF-1 (acidic FGF),
   transcript variant 1, Accession: NM_{—}000800, Gene ID: 15055546;
   transcript variant 2, Accession: NM_{—}033136, Gene ID: 15055540;
   transcript variant 3, Accession: NM_{—}033137, Gene ID: 15055544.
   20 siRNA candidates were selected:

| | | |
|---|---|---|
| # | Position | Sequence |

| | | |
|---|---|---|
| FGF-1-1 | 447-467 | AAGGCTGGAGGAGAACCATTA |
| FGF-1-2 | 214-234 | AAGCCCAAACTCCTCTACTGT |
| FGF-1-3 | 190-210 | AATCTGCCTCCAGGGAATTAC |
| FGF-1-4 | 114-134 | AAGCGCCACAAGCAGCAGCTG |
| FGF-1-5 | 484-504 | AAGAAGCATGCAGAGAAGAAT |
| FGF-1-6 | 539-559 | AACGCGGTCCTCGGACTCACT |
| FGF-1-7 | 460-480 | AACCATTACAACACCTATATA |
| FGF-1-8 | 97-117 | AAGCTCTTTAGTCTTGAAAGC |
| FGF-1-9 | 469-489 | AACACCTATATATCCAAGAAG |
| FGF-1-10 | 221-241 | AACTCCTCTACTGTAGCAACG |
| FGF-1-11 | 288-308 | AAGGGACAGGAGCGACCAGCA |
| FGF-1-12 | 487-507 | AAGCATGCAGAGAAGAATTGG |
| FGF-1-13 | 113-133 | AAAGCGCCACAAGCAGCAGCT |
| FGF-1-14 | 502-522 | AATTGGTTTGTTGGCCTCAAG |
| FGF-1-15 | 520-540 | AAGAAGAATGGGAGCTGCAAA |
| FGF-1-16 | 211-231 | AAGAAGCCCAAACTCCTCTAC |
| FGF-1-17 | 538-558 | AAACGCGGTCCTCGGACTCAC |
| FGF-1-18 | 526-546 | AATGGGAGCTGCAAACGCGGT |
| FGF-1-19 | 220-240 | AAACTCCTCTACTGTAGCAAC |
| FGF-1-20 | 424-444 | AATGAGGAATGTTTGTTCCTG |

### SS3.3.

FGFR2 gene: Human FGFR2,
   transcript variant 1, Accession: NM_{—}000141, Gene ID: 13186239;
   transcript variant 2, Accession: NM_{—}022969, Gene ID: 13186252;
   transcript variant 3, Accession: NM_{—}022970, Gene ID: 13186254.
   transcript variant 4, Accession: NM_{—}022971, Gene ID: 13186256;
   transcript variant 5, Accession: NM_{—}022972, Gene ID: 13186258;
   transcript variant 6, Accession: NM_{—}022973, Gene ID: 13186260.
   transcript variant 7, Accession: NM_{—}022974, Gene ID: 13186262;
   transcript variant 8, Accession: NM_{—}022975, Gene ID: 27754768;
   transcript variant 9, Accession: NM_{—}022976, Gene ID: 13186266.
   transcript variant 10, Accession: NM_{—}023028, Gene ID: 13186268;
   transcript variant 11, Accession: NM_{—}023029, Gene ID: 13186242;
   transcript variant 12, Accession: NM_{—}023030, Gene ID: 13186270.
   transcript variant 13, Accession: NM_{—}023031, Gene ID: 13186272;
   20 siRNA candidates were selected:

| | | |
|---|---|---|
| # | Position | Sequence |

| | | |
|---|---|---|
| FGFR2-1 | 1368-1388 | AAGCCGGACTGCCGGCAAATG |
| FGFR2-2 | 2610-2630 | AAGCCCTGTTTGATAGAGTAT |
| FGFR2-3 | 2088-2108 | AAGCAGTGGGAATTGACAAAG |
| FGFR2-4 | 2297-2317 | AAAGGCAACCTCCGAGAATAC |
| FGFR2-5 | 1753-1773 | AATCGCCTGTATGGTGGTAAC |
| FGFR2-6 | 2010-2030 | AATGGGAGTTTCCAAGAGATA |
| FGFR2-7 | 699-719 | AAGAGCCACCAACCAAATACC |
| FGFR2-8 | 2843-2863 | AAGCAGTTGGTAGAAGACTTG |
| FGFR2-9 | 1187-1207 | AAGCAGGAGCATCGCATTGGA |
| FGFR2-10 | 1082-1102 | AAGCGGCTCCATGCTGTGCCT |
| FGFR2-11 | 1557-1577 | AAGAGATTGAGGTTCTCTATA |
| FGFR2-12 | 1771-1791 | AACAGTCATCCTGTGCCGAAT |
| FGFR2-13 | 2762-2782 | AAGCCAGCCAACTGCACCAAC |
| FGFR2-14 | 1178-1198 | AAGGAGTTTAAGCAGGAGCAT |
| FGFR2-15 | 2151-2171 | AAGATGATGCCACAGAGAAAG |
| FGFR2-16 | 2745-2765 | AAGGACACAGAATGGATAAGC |
| FGFR2-17 | 1171-1191 | AAACGGGAAGGAGTTTAAGCA |
| FGFR2-18 | 1222-1242 | AAACCAGCACTGGAGCCTCAT |
| FGFR2-19 | 2732-2752 | AAGCTGCTGAAGGAAGGACAC |
| FGFR2-20 | 1556-1576 | AAAGAGATTGAGGTTCTCTAT |

### SS3.4.

FGFR1 gene: Human FGFR1
   transcript variant 1, Accession: NM_{—}000604, Gene ID: 13186232;
   transcript variant 2, Accession: NM_{—}015850, Gene ID: 13186250;
   transcript variant 3, Accession: NM_{—}023105, Gene ID: 13186233.
   transcript variant 4, Accession: NM_{—}023106, Gene ID: 13186235;
   transcript variant 5, Accession: NM_{—}023107, Gene ID: 13186237;
   transcript variant 6, Accession: NM_{—}023108, Gene ID: 13186240.
   transcript variant 7, Accession: NM_{—}023109, Gene ID: 13186244;
   transcript variant 8, Accession: NM_{—}023110, Gene ID: 13186246;
   transcript variant 9, Accession: NM_{—}023111, Gene ID: 13186248.
20 siRNA candidates were selected:

| | | |
|---|---|---|
| # | Position | Sequence |

| | | |
|---|---|---|
| FGFR1-1 | 2701-2721 | AACGGCCGACTGCCTGTGAAG |
| FGFR1-2 | 2275-2295 | AAGTCGGACGCAACAGAGAAA |
| FGFR1-3 | 2422-2442 | AAGGGCAACCTGCGGGAGTAC |
| FGFR1-4 | 2255-2275 | AAGTGGCTGTGAAGATGTTGA |
| FGFR1-5 | 2319-2339 | AATGGAGATGATGAAGATGAT |
| FGFR1-6 | 2237-2257 | AACCCAACCGTGTGACCAAAG |
| FGFR1-7 | 2887-2907 | AAGCCCAGTAACTGCACCAAC |
| FGFR1-8 | 1540-1560 | AACGTGGAGTTCATGTGTAAG |
| FGFR1-9 | 2236-2256 | AAACCCAACCGTGTGACCAAA |
| FGFR1-10 | 2332-2352 | AAGATGATCGGGAAGCATAAG |
| FGFR1-11 | 1153-1173 | AACACCAAACCAAACCGTATG |
| FGFR1-12 | 1303-1323 | AATGGCAAAGAATTCAAACCT |
| FGFR1-13 | 2905-2925 | AACGAGCTGTACATGATGATG |
| FGFR1-14 | 1636-1656 | AACCTGCCTTATGTCCAGATC |
| FGFR1-15 | 2857-2877 | AAGCTGCTGAAGGAGGGTCAC |
| FGFR1-16 | 1596-1616 | AAAGCACATCGAGGTGAATGG |
| FGFR1-17 | 2230-2250 | AAGGACAAACCCAACCGTGTG |
| FGFR1-18 | 2968-2988 | AAGCAGCTGGTGGAAGACCTG |
| FGFR1-19 | 2254-2274 | AAAGTGGCTGTGAAGATGTTG |
| FGFR1-20 | 1444-1464 | AACCACACATACCAGCTGGAT |

### SS3.5.

FGFR3 gene: Human FGFR3, Accession: M58051, Gene ID: 182568
   transcript variant 1, Accession: NM_{—}000142, Gene ID: 13112046;
   transcript variant 2, Accession: NM_{—}022965, Gene ID: 13112047;
   20 siRNA candidates were selected:

| | | |
|---|---|---|
| # | Position | Sequence |

| | | |
|---|---|---|
| FGFR3-1 | 1969-1989 | AACCTCGACTACTACAAGAAG |
| FGFR3-2 | 1627-1647 | AAGATGATCGGGAAACACAAA |
| FGFR3-3 | 1588-1608 | AAGGACCTGTCGGACCTGGTG |
| FGFR3-4 | 865-885 | AAGGTGTACAGTGACGCACAG |
| FGFR3-5 | 2263-2283 | AAGCAGCTGGTGGAGGACCTG |
| FGFR3-6 | 652-672 | AAGCTGCGGCATCAGCAGTGG |
| FGFR3-7 | 1540-1560 | AAGCCTGTCACCGTAGCCGTG |
| FGFR3-8 | 1571-1591 | AAGACGATGCCACTGACAAGG |
| FGFR3-9 | 1321-1341 | AACGCGTCCATGAGCTCCAAC |
| FGFR3-10 | 1297-1317 | AAGCGACAGGTGTCCCTGGAG |
| FGFR3-11 | 2191-2211 | AACTGCACACACGACCTGTAC |
| FGFR3-12 | 994-1014 | AAGGAGCTAGAGGTTCTCTCC |
| FGFR3-13 | 1570-1590 | AAAGACGATGCCACTGACAAG |
| FGFR3-14 | 982-1002 | AACACCACCGACAAGGAGCTA |
| FGFR3-15 | 1873-1893 | AAGTGCATCCACAGGGACCTG |
| FGFR3-16 | 331-351 | AATGCCTCCCACGAGGACTCC |
| FGFR3-17 | 1813-1833 | AAGGACCTGGTGTCCTGTGCC |
| FGFR3-18 | 2152-2172 | AAGCTGCTGAAGGAGGGCCAC |
| FGFR3-19 | 1723-1743 | AACCTGCGGGAGTTTCTGCGG |
| FGFR3-20 | 265-285 | AAGGATGGCACAGGGCTGGTG |

### SS3.6.

FGFR4 gene: Human FGFR4, Accession: L03840, Gene ID: 182570
   transcript variant 1, Accession: NM_{—}002011, Gene ID: 47524172;
   transcript variant 2, Accession: NM_{—}022963, Gene ID: 47524176;
   transcript variant 3, Accession: NM_{—}213647, Gene ID: 47524174;
   20 siRNA candidates were selected:

| | | |
|---|---|---|
| # | Position | Sequence |

| | | |
|---|---|---|
| FGFR4-1 | 726-746 | AAGGATGGACAGGCCTTTCAT |
| FGFR4-2 | 2403-2423 | AAGGTCCTGCTGGCCGTCTCT |
| FGFR4-3 | 1743-1763 | AAGCTGATCGGCCGACACAAG |
| FGFR4-4 | 1085-1105 | AAAGACTGCAGACATCAATAG |
| FGFR4-5 | 292-312 | AAGAGCAGGAGCTGACAGTAG |
| FGFR4-6 | 1657-1677 | AAGCCAGCACTGTGGCCGTCA |
| FGFR4-7 | 753-773 | AACCGCATTGGAGGCATTCGG |
| FGFR4-8 | 1833-1853 | AAGGGAAACCTGCGGGAGTTC |
| FGFR4-9 | 1392-1412 | AAGCTCTCCCGCTTCCCTCTG |
| FGFR4-10 | 1078-1098 | AAGTCCTAAAGACTGCAGACA |
| FGFR4-11 | 1692-1712 | AACGCCTCTGACAAGGACCTG |
| FGFR4-12 | 604-624 | AAGCACCCTACTGGACACACC |
| FGFR4-13 | 1086-1106 | AAGACTGCAGACATCAATAGC |
| FGFR4-14 | 1686-1706 | AAAGACAACGCCTCTGACAAG |
| FGFR4-15 | 666-686 | AACACCGTCAAGTTCCGCTGT |
| FGFR4-16 | 1454-1474 | AAGCTCATCCCTGGTACGAGG |
| FGFR4-17 | 984-1004 | AAGGTGTACAGCGATGCCCAG |
| FGFR4-18 | 1687-1707 | AAGACAACGCCTCTGACAAGG |
| FGFR4-19 | 1764-1784 | AACATCATCAACCTGCTTGGT |
| FGFR4-20 | 504-524 | AATCTCACCTTGATTACAGGT |

### SS4.1. Other Pathways I

| | | |
|---|---|---|
| HP BRCA2-A | AAGTCAACCACAGAGTCGTAT | 247-268 |
| HP BRCA2-B | AAGTAACGAGTGAGCCACGCT | 215-235 |
| NOXA-A | AAGTCGAGTGTGCTACTCAAC | 238-258 |
| NOX | AACTGAACTTCCGGCAGAAAC | 277-297 |
| Novel ZF Protein | AATGCGGAGAACACTAATTAT | 345-365 |
| Novel ZF Protein | AACTTCCATAAATGTGAAATC | 381-401 |
| NFAT4 | AAGTGATACTCCCGCCTCAGC | 726-746 |
| NFAT4 | AAGTAGCTGGCACTACGGGCA | 752-772 |
| Co-factor of SP1 | AATCAGGTTCCAATGTGATGA | 200-221 |
| Co-factor of SP1 | AAGGCTTAGCTCCCAAGCCTC | 145-165 |
| Ets2 Repressor | AAGGCAGATCCAGCTGTGGCA | 194-214 |
| Ets2 Repressor | AAGCCAGAGTCGTCCCCTGGC | 171-191 |
| PKC related | AAGTCTTCCGTTTTCTGAGAA | 69-89 |
| PKC related | AATGGTGCAGCAGAAATTGGA | 126-136 |
| PKC eta | AAGAAGGGCCACCAGCTGCTG | 269-289 |
| PKC eta | AACGTCACCGACGGCGGCCAC | 389-409 |
| Mitochondrial F0 | AACCTCGGGCAGAAGAGGAGA | 164-184 |
| Mitochondrial F0 | AACTGAAACGGATTGCCAGAG | 211-231 |
| Bcl-2 TF | AAGAAGCGATACAGGTGTCGT | 91-111 |
| Bcl-2 TF | AAGGTCTCGTAGTAGAGATCG | 126-146 |
| Bcl-2 A1 | AACCTGGATCAGGTCCAAGCA | 257-277 |
| Bcl-2 A1 | AATCTGAAGTCATGCTTGGAC | 334-354 |
| RAP1 | AACAGAGGAGGACTACATTCC | 267-287 |
| RAP1 | AACCACGAAATCACCAGCATC | 379-399 |

### SS5.1. Other Pathways II

| | | |
|---|---|---|
| EGFR-RP-A | AK026010 | AAGCTGGACATTCCCTCTGCG |
| EGFR-RP-B | | AAGAGCCCAGCTTCCTGCAGC |
| ENDOPLASMIN 94-A | AK025862 | AACTGTTGAGGAGCCCATGGA |
| ENDOPLASMIN 94-B | | AATCTGATGATGAAGCTGCAG |
| FOLATE BP-A | AF000381 | AACCGCGGTCCTATTCCATTA |
| FOLATE BP-B | | AACACTCCAATTTTTCAAAGT |
| A-RAF-A | U33821 | AAGAGTTACCTTCCTAATGCA |
| A-RAF-B | | AAGATTGGGTTGGTATATTCA |
| NOVEL-1-A | NM_017873 | AATCCTTGTTCTCACTGAGCT |
| NOVEL-1-B | | AAGATGGCTGAGCTGGGGCTG |
| EGF FACTOR 8-A | NM_005928 | AACCCCTGCCACAACGGTGGT |
| EGF FACTOR 8-B | | AACCACTGTGAGACGAAATGT |
| APRIL-A | AK090698 | AACTGCCCCAGCGATCTCTGC |
| APRIL-B | | AACCTAATTCTCCTGAGGCTG |
| PGF PRECURSOR-A | AK023843 | AAGAGTGACACTGTGGCTTCC |
| PGF PRECURSOR-B | | AATGGGCTGAGCTGCTGCTCC |

SS6, TNF pathway
INF pathway

### SS6.1.

TNF gene: human TNF (synonyms: DIF, TNFA, TNFSF2, TNF-alpha), Accession:
   NM_{—}000594, Gene ID: 25952110
   10 siRNA candidates were selected:

| | | |
|---|---|---|
| # | Position | Sequence |

| | | |
|---|---|---|
| hTNF-1 | 428-448 | AAGCCTGTAGCCCATGTTGTA |
| hTNF-2 | 512-532 | AATGGCGTGGAGCTGAGAGAT |
| hTNF-3 | 671-691 | AACCTCCTCTCTGCCATCAAG |
| hTNF-4 | 533-553 | AACCAGCTGGTGGTGCCATCA |
| hTNF-5 | 731-751 | AAGCCCTGGTATGAGCCCATC |
| hTNF-6 | 497-517 | AATGCCCTCCTGGCCAATGGC |
| hTNF-7 | 779-899 | AAGGGTGACCGACTCAGCGCT |
| hTNF-8 | 181-201 | AAGCATGATCCGGGACGTGGA |
| hTNF-9 | 665-685 | AAGGTCAACCTCCTCTCTGCC |
| hTNF-10 | 180-200 | AAAGCATGATCCGGGACGTGG |

### SS6.2.

hTNFR1 gene: human TNF receptor, 1A (synonyms: TNFRSF1A, FPF, p55, p60, TBP1, TNF-R, TNFAR, TNFR1,p55-R, CD120a, TNFR55, TNFR60, TNF-R-I, TNF-R55, MGC19588), Accession: NM_{—}001065, Gene ID: 23312372
   20 siRNA candidates were selected:

| | | |
|---|---|---|
| # | Position | Sequence |

| | | |
|---|---|---|
| hTNFR1-1 | 666-686 | AAGAACCAGTACCGGCATTAT |
| hTNFR1-2 | 1005-1025 | AAGCTCTACTCCATTGTTTGT |
| hTNFR1-3 | 1320-1340 | AAGCCACAGAGCCTAGACACT |
| hTNFR1-4 | 841-861 | AAAGCCTGGAGTGCACGAAGT |
| hTNFR1-5 | 472-492 | AAGGAACCTACTTGTACAATG |
| hTNFR1-6 | 714-734 | AATTGCAGCCTCTGCCTCAAT |
| hTNFR1-7 | 605-625 | AATGGGTCAGGTGGAGATCTC |
| hTNFR1-8 | 669-689 | AACCAGTACCGGCATTATTGG |
| hTNFR1-9 | 471-491 | AAAGGAACCTACTTGTACAAT |
| hTNFR1-10 | 462-482 | AAGTGCCACAAAGGAACCTAC |
| hTNFR1-11 | 604-624 | AAATGGGTCAGGTGGAGATCT |
| hTNFR1-12 | 810-830 | AACGAGTGTGTCTCCTGTAGT |
| hTNFR1-13 | 888-908 | AAGGGCACTGAGGACTCAGGC |
| hTNFR1-14 | 809-829 | AAACGAGTGTGTCTCCTGTAG |
| hTNFR1-15 | 991-1011 | AACGGTGGAAGTCCAAGCTCT |
| hTNFR1-16 | 768-788 | AACACCGTGTGCACCTGCCAT |
| hTNFR1-17 | 732-752 | AATGGGACCGTGCACCTCTCC |
| hTNFR1-18 | 1089-1109 | AACCCAAGCTTCAGTCCCACT |
| hTNFR1-19 | 476-496 | AACCTACTTGTACAATGACTG |
| hTNFR1-20 | 444-464 | AATTCGATTTGCTGTACCAAG |

### SS6.3.

hTNFR2 gene: human TNF receptor, 1B (synonyms: TNFRSF1B, p75, TBPII, TNFBR, TNFR2, CD120b, TNFR80, TNF-R75, p75TNFR, TNF-R-II), Accession: NM_{—}001066, Gene ID: 23312365. 20 siRNA candidates were selected:

| | | |
|---|---|---|
| # | Position | Sequence |

| | | |
|---|---|---|
| hTNFR2-1 | 844-864 | AAGGGAGCACTGGCGACTTCG |
| hTNFR2-2 | 957-977 | AAGCCCTTGTGCCTGCAGAGA |
| hTNFR2-3 | 412-432 | AAGCCTGCACTCGGGAACAGA |
| hTNFR2-4 | 1362-1382 | AAGGAGGAATGTGCCTTTCGG |
| hThFR2-5 | 294-314 | AAGACCTCGGACACCGTGTGT |
| hTNFR2-6 | 351-371 | AACTGGGTTCCCGAGTGCTTG |
| hTNFR2-7 | 784-804 | AACCCAGCACTGCTCCAAGCA |
| hTNFR2-8 | 1301-1321 | AATGGGAGACACAGATTCCAG |
| hTNFR2-9 | 979-1099 | AAGCCAAGGTGCCTCACTTGC |
| hTNFR2-10 | 914-934 | AATAGGAGTGGTGAACTGTGT |
| hTNFR2-11 | 1227-1247 | AATGTCACCTGCATCGTGAAC |
| hTNFR2-12 | 600-620 | AACACGACTTCATCCACGGAT |
| hTNFR2-13 | 1288-1308 | AAGCCAGCTCCACAATGGGAG |
| hTNFR2-14 | 432-452 | AACCGCATCTGCACCTGCAGG |
| hTNFR2-15 | 984-1004 | AAGGTGCCTCACTTGCCTGCC |
| hTNFR2-16 | 800-820 | AAGCACCTCCTTCCTGCTCCC |
| hTNFR2-17 | 954-974 | AAGAAGCCCTTGTGCCTGCAG |
| hTNFR2-18 | 1245-1265 | AACGTCTGTAGCAGCTCTGAC |
| hTNFR2-19 | 1369-1389 | AATGTGCCTTTCGGTCACAGC |
| hTNFR2-20 | 776-796 | AACTCCAGAACCCAGCACTGC |

### SS6.4.

| | |
|---|---|
| mouse IL-1b | AGGCTCCGAGATGAACAACAA |
| mouse IL-1b | TACCTGTCCTGTGTAATGAAA |
| mouse IL-1r | ACCATCGAGGTTACTAATGAA |
| mouse IL-1r | TCGGAATATCTCCCATCATAA |
| mouse IL-1a | TCGGGAGGAGACGACTCTAAA |
| mouse IL-1a | CCAGAGTGATTTGAGATACAA |
| mouse IL-1r2 | CACGTTTATCTCGGCTGCTTA |
| mouse IL-1r2 | AAGACTGATAGTCCCGTGCAA |
| mouse TNF receptor a | AAGGAAAGTATGTCCATTCTA |
| mouse TNF receptor a | CCGCAACGTCCTGACAATGCA |
| mouse TNF receptor b | CCAGGTTGTCTTGACACCCTA |
| mouse TNF receptor b | CTGGCTATTCCCGGAAATGCA |
| mouse TNF | CACGTCGTAGCAAACCACCAA |
| mouse TNF | CAGCCGATTTGCTATCTCATA |

1. A composition comprising at least one dsRNA oligonucleotide and a pharmaceutical carrier, wherein upon administration to a subject suffering from an ocular disease associated with neovascularization or angiogenesis said dsRNA inhibits expression of a gene associated with neovascularization or angiogenesis in an ocular disease.

2. The composition according to claim 1 where the pharmaceutical carrier is selected from the group of a polymer, lipid, or micelle.

3. The composition according to claim 1 wherein said ocular disease is selected from the group consisting of stromal keratitis, uveitis, rubeosis, conjunctivitis, keratitis, blepharitis, sty, chalazion, iritis, macular degeneration, and retinopathy.

4. A composition according to claim 1 wherein said dsRNA inhibits expression of a gene selected from the group of pro-inflammatory pathway genes, pro-angiogenesis pathway genes, pro-cell proliferation pathway genes, and viral infectious agent genome RNA, and viral infectious agent genes.

5. A composition according to claim 4 comprising at least two dsRNA molecules, where each dsRNA molecule inhibits expression of a gene selected from the group of pro-inflammatory pathway genes, pro-angiogenesis pathway genes, pro-cell proliferation pathway genes, and viral infectious agent genome RNA, and viral infectious agent genes.

6. A composition according to claim 5 comprising at least three dsRNA molecules wherein at least one dsRNA molecule inhibits expression of VEGF, at least one dsRNA molecule inhibits expression of VEGF R1, and at least one dsRNA molecule inhibits expression of VEGF R2

7. A composition according to claim 5 comprising at least two dsRNA molecules wherein at least one dsRNA molecule inhibits expression of basic FGF and at least one dsRNA molecule inhibits expression of FGF R.

8. A composition according to claim 5 wherein said dsRNA molecules inhibit expression of one or more VEGF pathway genes, FGF pathway genes, or a combination thereof.

9. A composition according to claim 5 wherein said dsRNA molecules inhibit expression one or more pro-angiogenesis genes, pro-inflammatory genes, or a combination thereof

10. A composition according to claim 5 wherein said dsRNA molecules inhibit expression of one or more pro-angiogenesis genes, herpes simplex virus genes, or a combination thereof.

11. A composition according to claim 5 wherein said dsRNA molecules inhibit expression of one or more pro-angiogenesis genes, endothelial cell proliferation genes, or a combination thereof.

12. A composition according to claim 5 wherein said dsRNA molecules inhibit expression of one or more pro-inflammation genes, herpes simplex virus genes, or a combination thereof.

13. A composition according to claim 5 comprising at least three dsRNA molecules that inhibit expression of at least two or more genes.

14. A composition according to claim 13 wherein said genes encode VEGF, VEGF R1, and VEGF R2.

15. A composition according to claim 13 wherein said genes encode expression of basic FGF and FGF R.

16. A composition according to claim 13 wherein said genes encode VEGF pathway genes, FGF pathway genes, or a combination thereof.

17. A composition according to claim 13 wherein said genes are pro-angiogenesis genes, pro-inflammatory genes, or a combination thereof.

18. A composition according to claim 13 wherein said genes are pro-angiogenesis genes, herpes simplex virus genes, or a combination thereof.

19. A composition according to claim 13 wherein said genes are pro-angiogenesis genes, endothelial cell proliferation genes, or a combination thereof.

20. A composition according to claim 13 wherein said genes are pro-inflammation genes, herpes simplex virus genes, or a combination thereof.

21. A composition according to claim 2 where said carrier is selected from the group consisting of polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer.

22. The composition according to claim 1 wherein said dsRNA molecule is a dsRNA oligonucleotide.

23. A method for treating ocular disease in a subject, wherein said disease is characterized at least in part by neovascularization, comprising administering to said subject a composition comprising a dsRNA oligonucleotide and a pharmaceutically acceptable carrier, wherein said dsRNA oligonucleotide inhibits expression of a gene that promotes ocular neovascularization in said subject.

24. The method according to claims 23, wherein said ocular disease is in at least the anterior of the eye.

25. A method according to claim 23 wherein said composition is administered at a site distal to the eye selected from the group of subconjunctival, intravenous, and subcutaneous.

26. A method according to claim 23 wherein said composition is administered topically to the eye.

27. A method according to claim 23 where said pharmaceutical carrier is selected from the group of a polymer, lipid, or micelle.

28. A method according to claim 23 where the ocular disease is selected from the group of stromal keratitis, uveitis, rubeosis, conjunctivitis, keratitis, blepharitis, sty, chalazion, iritis, macular degeneration, and retinopathy.

29. A method according to claim 23 wherein said the dsRNA inhibits expression of at least one gene selected from the group of pro-inflammatory pathway genes, pro-angiogenesis pathway genes, pro-cell proliferation pathway genes, and viral infectious agent genome RNA, and viral infectious agent genes.

30. A method according to claim 23 wherein said dsRNA inhibits expression of more than one gene.

31. A method according to claim 30 wherein said dsRNA inhibits expression of VEGF, VEGF R1, and VEGF R2.

32. A method according to claim 30 wherein said dsRNA inhibits expression of basic FGF and FGF R.

33. A method according to claim 30 wherein said dsRNA inhibits expression of VEGF pathway genes, FGF pathway genes, or a combination

34. A method according to claim 30 wherein said dsRNA inhibits expression of pro-angiogenesis genes, pro-inflammatory genes, or a combination thereof.

35. A method according to claim 30 wherein said dsRNA inhibits expression of pro-angiogenesis genes, herpes simplex virus genes, or a combination thereof.

36. A method according to claim 30 wherein said dsRNA inhibits expression of pro-angiogenesis genes, endothelial cell proliferation genes, or a combination thereof.

37. A method according to claim 30 wherein said dsRNA inhibits expression of pro-inflammation genes, herpes simplex virus genes, or a combination thereof.

38. A method according to claim 30 wherein said dsRNA inhibits expression of more than two genes.

39. A method according to claim 38 wherein said dsRNA inhibits expression of VEGF, VEGF R1, and VEGF R2.

40. A method according to claim 38 wherein said dsRNA inhibits expression of basic FGF and FGF R.

41. A method according to claim 38 wherein said dsRNA inhibits expression of VEGF pathway genes, FGF pathway genes, or a combination thereof.

42. A method according to claim 38 wherein said dsRNA inhibits expression of pro-angiogenesis genes, pro-inflammatory genes, or a combination thereof.

43. A method according to claim 38 wherein said dsRNA inhibits expression of pro-angiogenesis genes, herpes simplex virus genes, or a combination thereof.

44. The method according to claim 38 wherein said dsRNA inhibits expression of pro-angiogenesis genes, endothelial cell proliferation genes, or a combination thereof.

45. The method according to claim 38 wherein said dsRNA inhibits expression of pro-inflammation genes, herpes simplex virus genes, or a combination thereof.

46. A method according to claim 2 wherein said carrier is selected from the group of polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer.

47. The method according to claim 23 wherein said subject is a human.
